(19) United States
(12) Patent Application Publication
(10) Pub. No.: US 2025/0352674 A1
(43) Pub. Date: Nov. 20, 2025

(54) COMPOSITIONS AND METHODS FOR MODULATING NEURONAL EXCITABILITY

(71) Applicants: The Broad Institute, Inc., Cambridge, MA (US); President and Fellows of Harvard College, Cambridge, MA (US); Massachusetts Institute of Technology, Cambridge, MA (US)

(72) Inventors: Jia Liu, Cambridge, MA (US); Xiao Wang, Cambridge, MA (US)

(73) Assignees: The Broad Institute, Inc., Cambridge, MA (US); President and Fellows of Harvard College, Cambridge, MA (US); Massachusetts Institute of Technology, Cambridge, MA (US)

(21) Appl. No.: 19/228,209

(22) Filed: Jun. 4, 2025

Related U.S. Application Data
(63) Continuation of application No. PCT/US2023/082553, filed on Dec. 5, 2023.
(60) Provisional application No. 63/430,617, filed on Dec. 6, 2022.

(51) Int. Cl. A61K 48/00; A61K 31/785; A61K 41/00; A61P 25/00; C07K 14/415; C12N 15/86

(52) U.S. Cl. CPC A61K 48/0058; A61K 31/785; A61K 41/00; A61P 25/00; C07K 14/415; C12N 15/86; C12N 2750/14143

(57) ABSTRACT

The invention features compositions and methods for treating diseases or disorders associated with undesirable neuronal excitability (e.g., neurodegenerative disease, such as Parkinson's disease or Huntington's disease; or chronic pain, or epilepsy).

### CROSS REFERENCE TO RELATED APPLICATIONS

This application is a continuation under 35 U.S.C. § 111(a) of PCT International Patent Application No. PCT/US2023/082553, filed Dec. 5, 2023, designating the United States and published in English, which claims priority to and the benefit of U.S. Provisional Application No. 63/430,617, filed Dec. 6, 2022, the entire contents of each of which are incorporated by reference herein.

### STATEMENT OF RIGHTS TO INVENTIONS MADE UNDER FEDERALLY SPONSORED RESEARCH

This invention was made with government support under grant No. FA9550-22-1-0228 awarded by the Air Force Office of Scientific Research and grant No. DMR2011754 awarded by the National Science Foundation. The government has certain rights in the invention.

### BACKGROUND OF THE INVENTION

The intrinsic excitability of neurons is governed by two membrane properties: membrane conductivity and capacitance. Electrical, optogenetic, and pharmacological manipulations can transiently change membrane properties through manipulations of ion channels. In particular, conventional optogenetic stimulation harvests optical-driven ion channels (e.g., channelrhodopsin-2 (ChR2) and halorhodopsin (NpHR)), enabling millisecond-timescale, genetically-targeted, all-optical excitation and inhibition of living neurons. In contrast to transiently switching ion channels, naturally occurring neuron development and myelination processes suggest that modulating membrane capacitance is another effective way of manipulating neuron intrinsic excitability during brain development, learning, and aging. Increasing or decreasing the membrane capacitance can decrease or increase the cellular excitability and the velocity of action potential propagation.

Recent advances in materials science and nanotechnology have shown that the incorporation of miniaturized electrically functional materials and components onto cellular membranes can modulate the membrane capacitance, changing the intrinsic cellular activities in vitro, which can potentially alter the neuron excitability in a long-term manner. However, these techniques do not enable genetically targeted specificity in neuronal circuits, in part due to the difficulty of incorporating prefabricated nanomaterials into biological systems in a cell type- or subcellular-specific manner. In vivo synthesis of functional nanomaterials has recently emerged as a promising alternative strategy for the integration of nanomaterials with living systems, often providing greater control over the location and integration of materials at the cellular level. There remains a need for strategies to increase or decrease membrane excitability with increased specificity while minimizing exposure to toxic side reactions.

### SUMMARY OF THE INVENTION

The invention features compositions and methods for treating diseases or disorders associated with undesirable neuronal excitability (e.g., neurodegenerative disease, such as Parkinson's disease or Huntington's disease; or chronic pain, or epilepsy).

In one aspect, the invention provides a method for modulating neural activity using optogenetic polymerization and assembly of electroactive polymers on specified cellular membranes, the method involving expressing in a neuronal cell a vector comprising a mini Singlet Oxygen Generator (miniSOG) in the presence of monomers of 3,3′-diaminobenzidine (DAB) or aniline and N-phenyl-p-phenylenediamine; and irradiating at least a portion of the neuronal cell to induce polymerization of poly(3,3′-diaminobenzidine) or polyaniline, thereby modulating the neuronal activity.

In another aspect, the invention provides a method for increasing current injection-evoked action potential firing in response to depolarizing stimuli, the method involving expressing in a neuronal cell a vector comprising a mini Singlet Oxygen Generator (miniSOG) in the presence of monomers of 3,3′-diaminobenzidine; and irradiating at least a portion of the neuronal cell to induce polymerization of poly(3,3′-diaminobenzidine), thereby increasing current injection-evoked action potential firing in response to depolarizing stimuli.

In another aspect, the invention provides a method for decreasing action potential firing in response to depolarizing stimuli, the method involving expressing in a neuronal cell a vector comprising a mini Singlet Oxygen Generator (miniSOG) in the presence of monomers of aniline and N-phenyl-p-phenylenediamine; and irradiating at least a portion of the neuronal cell to induce polymerization of poly(3,3′-diaminobenzidine), thereby decreasing action potential firing in response to depolarizing stimuli.

In another aspect, the invention provides an adeno-associated viral expression vector (AAV) comprising a CAG or human synapsin promoter driving expression of a miniSOG fused to a T2A ribosome skipping sequence.

In another aspect, the invention provides a neuronal cell comprising the AAV of claim 19.

In another aspect, the invention provides a method for modulating neural activity using optogenetic polymerization and assembly of electroactive polymers on specified cellular membranes, the method involving expressing in a neuronal cell an adeno-associated viral expression vector (AAV) comprising a CAG or human synapsin promoter driving expression of a miniSOG fused to a T2A ribosome skipping sequence in the presence of monomers of 3,3′-diaminobenzidine or aniline and N-phenyl-p-phenylenediamine; and
(b) irradiating the neuronal cell to induce polymerization of poly(3,3′-diaminobenzidine) or polyaniline, thereby modulating the neuronal activity.
   In another aspect, the invention provides a method for increasing current injection-evoked action potential firing in response to depolarizing stimuli, the method involving
(a) expressing in a neuronal cell an adeno-associated viral expression vector (AAV) comprising a CAG or human synapsin promoter driving expression of a miniSOG fused to a T2A ribosome skipping sequence in the presence of monomers of 3,3′-diaminobenzidine; and
(b) irradiating at least a portion of the neuronal cell to induce polymerization of poly(3,3′-diaminobenzidine), thereby increasing current injection-evoked action potential firing in response to depolarizing stimuli.
   In another aspect, the invention provides a method for decreasing action potential firing in response to depolarizing stimuli, the method comprising
(a) expressing in a neuronal cell a vector comprising an adeno-associated viral expression vector (AAV) comprising a CAG or human synapsin promoter driving expression of a miniSOG fused to a T2A ribosome skipping sequence in the presence of monomers of aniline and N-phenyl-p-phenylenediamine; and
(b) irradiating at least a portion of the neuronal cell to induce polymerization of poly(3,3′-diaminobenzidine), thereby decreasing action potential firing in response to depolarizing stimuli.

In another aspect, the invention provides a method for modulating neural activity in a subject having a disorder associated with undesirable neural activity, the method comprising
(a) administering to a neuronal cell of the subject a vector comprising a mini Singlet Oxygen Generator (miniSOG) in the presence of monomers of 3,3′-diaminobenzidine or aniline and N-phenyl-p-phenylenediamine; and
(b) irradiating at least a portion of the neuronal cell to induce polymerization of poly(3,3′-diaminobenzidine) or polyaniline, thereby modulating the neuronal activity of the subject.

In another aspect, the invention provides a method for modulating neural activity in a subject having a disorder associated with undesirable neural activity, the method comprising
(a) administering to a neuronal cell of the subject an adeno-associated viral expression vector (AAV) comprising a CAG or human synapsin promoter driving expression of a miniSOG fused to a T2A ribosome skipping sequence in the presence of monomers of 3,3′-diaminobenzidine or aniline and N-phenyl-p-phenylenediamine; and
(b) irradiating the neuronal cell to induce polymerization of poly(3,3′-diaminobenzidine) or polyaniline, thereby modulating the neuronal activity of the subject.

In another aspect, the invention provides a kit for use in any of the above methods, the kit comprising a vector comprising a mini Singlet Oxygen Generator (miniSOG) and monomers of 3,3′-diaminobenzidine and/or aniline and N-phenyl-p-phenylenediamine.

In various embodiments of any of the above aspects or any other aspect of the invention delineated herein, the method provides for the spatiotemporal control of polymerization. In various embodiments of any of the above aspects, the method provides for photopolymerization of DAB at nanometer-level spatial resolution. In various embodiments of any of the above aspects, spatial control is at the subcellular level. In various embodiments of any of the above aspects, the method provides for optical control of polymer assembly on or within the cell membrane. In various embodiments of any of the above aspects, the miniSOG produces increased levels of singlet oxygen relative to other reactive oxygen species (ROS). In various embodiments of any of the above aspects, the method does not reduce neuron viability. In various embodiments of any of the above aspects, the method alters neuronal excitability. In various embodiments of any of the above aspects, the miniSOG is expressed under the control of a CAG promoter. In various embodiments of any of the above aspects, the vector is a viral vector. In various embodiments of any of the above aspects, the viral vector is an an adeno-associated viral expression vector (AAV) vector. In various embodiments of any of the above aspects, the irradiation is at a wave length of between about 425-500 nm. In various embodiments of any of the above aspects, the irradiation is at about 475 nm. In various embodiments of any of the above aspects, the irradiation is for about 5-8 minutes. In various embodiments of any of the above aspects, the irradiation is for about 9-15 minutes. In various embodiments of any of the above aspects, the neuron is in vitro or in vivo. In various embodiments of any of the above aspects, the neuron is a cell of the central or peripheral nervous system. In various embodiments of any of the above aspects, the neuron is a motor neuron or sensory neuron. In various embodiments of any of the above aspects, the method provides for photopolymerization of DAB at nanometer-level spatial resolution. In various embodiments of any of the above aspects, spatial control is at the subcellular level. In various embodiments of any of the above aspects, the method provides long term alterations in the electrophysiology of the neuron. In various embodiments of any of the above aspects, the electrophysiological changes last for at least about 1 month. In various embodiments of any of the above aspects, the electrophysiological changes last for at least about 1-3 weeks. In various embodiments of any of the above aspects, the electrophysiological changes last for at least about 1-3 days. In various embodiments of any of the above aspects, the method treats the disorder or ameliorates at least one symptom of the disorder. In various embodiments of any of the above aspects, the method provides for the spatiotemporal control of polymerization. In various embodiments of any of the above aspects, the neuron is irradiated in the presence of aniline and N-phenyl-p-phenylenediamine. In various embodiments of any of the above aspects, the disorder is a neurodegenerative disease. In various embodiments of any of the above aspects, the disorder is chronic pain or epilepsy. In various embodiments of any of the above aspects, the electrophysiological changes last for at least about 1-3 weeks. In various embodiments of any of the above aspects, the electrophysiological changes last for at least about 1-3 days. In various embodiments of any of the above aspects, the vector is an adeno-associated viral expression vector (AAV). In various embodiments of any of the above aspects, the vector comprises a CAG or human synapsin promoter driving expression of the miniSOG fused to a T2A ribosome skipping sequence.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a polypeptide or nucleic acid molecule, or active fragments thereof, or a small molecule chemical compound. In embodiments, the agent is an electroactive polymer whose polymerization is induced using optogenetic polymerization.

By “ameliorate” is meant decrease, suppress, attenuate, diminish, arrest, or stabilize the development or progression of a disease. In embodiments, the disease is a neurodegenerative disorder.

By “alteration” is meant a change (increase or decrease) in the expression levels, structure, or activity of a cell, a gene or polypeptide as detected by standard art known methods such as those described herein. As used herein, an alteration includes a 10% change in expression levels, preferably a 25% change, more preferably a 40% change, and most preferably a 50% or greater change in expression levels.

By “aniline” is meant a compound having the chemical formula C₆H₅NH₂ and/or corresponding to CAS Number 62-53-3. Aniline may also be known as Benzenamine. An exemplary chemical structure for aniline may be found below:

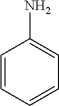

By “3,3′-diaminobenzidine” or “DAB” is meant a compound having the chemical formula (C₆H₃(NH₂)₂)₂ and/or corresponding to CAS Number 91-95-2. 3,3′-diaminobenzidine may also be known as [1,1′-Biphenyl]-3,3′,4,4′-tetramine. An exemplary chemical structure for 3,3′-diaminobenzidine may be found below:

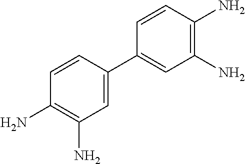 [SMILES: Nc1ccc(-c2ccc(N)c(N)c2)cc1N]

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “decreases” is meant a reduction by at least about 5% relative to a reference level. A decrease may be by 5%, 10%, 15%, 20%, 25% or 50%, or even by as much as 75%, 85%, 95% or more and any intervening percentages.

“Detect” refers to identifying the presence, absence or amount of the analyte to be detected. In embodiments, the analyte is an electroactive polymer.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. In an embodiment, the disease is a neurodegenerative disease or a disorder characterized by undesirable neuronal activity.

By “disorder characterized by undesirable neuronal activity” is meant any increase or decrease in neuronal activity in a subject that disrupts the normal electrophysiology of the subject. Examples of such disorders include, but are not limited to, epilepsy, Parkinson's disease, Huntington's disease, chronic pain and other related disorders.

“Contacting” is used in accordance with its plain ordinary meaning and refers to the process of allowing at least two distinct species (e.g. chemical compounds including biomolecules, or cells) to become sufficiently proximal to react, interact, affect or physically touch. It should be appreciated, however, that the resulting reaction product can be produced directly from a reaction between the added reagents or from an intermediate from one or more of the added reagents, which can be produced in the reaction mixture. Contacting may include allowing two species to react, interact, or physically touch, wherein the two species may be a recombinant viral particle as described herein and a cell. In embodiments, the two species are an ultrasound contrast agent that is exposed to ultrasound and a cell.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

By “effective amount” is meant the amount of a vector and/or monomers described herein required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

A “host cell” or “cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “miniSOG polypeptide” is meant a protein having at least about 85% amino acid sequence identity to Genbank Reference Sequence No. AGE44112.1 or a fragment thereof having singlet oxygen generator activity. An exemplary miniSOG amino acid sequence follows:

| | |
|---|---|
| 1 | MEKSFVITDP RLPDNPIIFA SDGFLELTEY SREEILGRNG RFLQGPETDQ ATVQKIRDAI |
| 61 | RDQREITVQL INYTKSGKKF WNLLHLQPMR DQKGELQYFI GVQLDG. |

miniSOG is described by Shu et al., PLoS Biol. 2011 April; 9(4): e1001041, which is incorporated herein by reference in its entirety.

By “miniSOG polynucleotide” is meant a polynucleotide sequence encoding a miniSOG polypeptide. An exemplary miniSOG polynucleotide sequence is provided at Genbank Reference Sequence No. JX999997.1, which is reproduced below:

| | |
|---|---|
| 1 | atggaaaaga gctttgtgat taccgatccg cgcctgccag acaacccgat cattttcgcg |
| 61 | agcgatggct ttctggagtt aaccgaatat tctcgtgagg aaattctggg tcgcaatggc |
| 121 | cgtttcttgc agggtccgga aacggatcaa gccaccgtgc agaaaatccg cgatgcgatt |
| 181 | cgtgaccaac gcgaaatcac cgttcagctg attaactata cgaaaagcgg caagaaattt |
| 241 | tggaacttac tgcatctgca accgatgcgc gatcagaaag gcgaattgca atatttcatt |
| 301 | ggtgtgcagc tggatggcta g |

By “N-phenyl-p-phenylenediamine” is meant a compound having the chemical formula C₆H₅NHC₆H₄NH₂ and/or corresponding to CAS Number 101-54-2. N-phenyl-p-phenylenediamine may also be known as 4-Aminodiphenylamine. An exemplary chemical structure for N-phenyl-p-phenylenediamine may be found below:

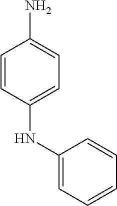 [SMILES: Nc1ccc(Nc2ccccc2)cc1]

By “operably linked” refers to a functional linkage between a regulatory sequence and a coding sequence, where a first polynucleotide is positioned adjacent to a second polynucleotide that directs transcription of the first polynucleotide when appropriate molecules (e.g., transcriptional activator proteins) are bound to the second polynucleotide. The described components are therefore in a relationship permitting them to function in their intended manner. For example, placing a coding sequence under regulatory control of a promoter means positioning the coding sequence such that the expression of the coding sequence is controlled by the promoter.

By “poly(3,3′-diaminobenzidine)” or “PDAB” is meant a polymer comprising monomers of 3,3′-diaminobenzidine.

By “polyaniline” or “PANI” is meant a polymer comprising monomers of aniline.

By “portion” is meant a fragment of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 16, 17, 18, 19, 20, or 21 nucleotides.

By “positioned for expression” is meant that the polynucleotide of the disclosure (e.g., a DNA molecule) is positioned adjacent to a DNA sequence that directs transcription and translation of the sequence (i.e., facilitates the production of, for example, a recombinant microRNA molecule described herein).

The term “promoter” as used herein refers to a sequence of DNA that directs the expression (transcription) of a gene. A promoter may direct the transcription of a prokaryotic or eukaryotic gene. A promoter may be “inducible”, initiating transcription in response to an inducing agent or, in contrast, a promoter may be “constitutive”, whereby an inducing agent does not regulate the rate of transcription. A promoter may be regulated in a tissue-specific or tissue-preferred manner, such that it is only active in transcribing the operable linked coding region in a specific tissue type or types. Exemplary promoters include the CAG promoter and/or the human synapsin promoter, or any other promoter that directs expression in a neuron of interest.

By “reactive oxygen species” is meant a class of chemically-reactive molecules containing at least one oxygen atom. Reactive oxygen species include oxygen free radicals, such as, for example, superoxide anion radical, hydroxyl radical, hydroperoxyl radical, and singlet oxygen, as well as molecules lacking oxygen free radicals, such as, for example, hydrogen peroxide, ozone, and peroxynitrite.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

By “reduces” is meant a negative alteration of at least 10%, 25%, 50%, 75%, or 100%.

By “reference” is meant a standard or control condition. For example, an untreated cell, tissue, or organ that is used as a reference. In one embodiment, a reference is a cell of normal electrophysiology that is used as the basis for comparison relative to a cell expressing a vector comprising a miniSOG alone or in combination with monomers described herein, which are subsequently irradiated to form polymers.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “transformed cell” is meant a cell into which (or into an ancestor of which) has been introduced, by means of recombinant DNA techniques, a polynucleotide molecule encoding (as used herein) a polypeptide of the invention.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or 15 functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

By “vector” is meant a nucleic acid molecule, for example, a plasmid, cosmid, virus, or bacteriophage that is capable of replication in a host cell. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, and enhancers.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1J provide schematics, images, and bar graphs showing optogenetic polymerization and assembly of electrically functional materials in cells. FIG. 1A provides a schematic showing that photosensitizer proteins are expressed in the cytosol of the genetically specified neuron types. Upon light irradiation and introduction of polymer monomers, photosensitizer proteins locally polymerize and assemble conductive or insulating polymers in the cell and on the cell membrane, modulating the membrane capacitance and excitability. FIGS. 1B-1C provide images showing merged confocal fluorescence (left) and transmitted light (TL, right) images of fixed HEK293T cells co-expressing dTomato and miniSOG after 7 minutes of irradiation in the presence of 1 mM DAB (labeled as PDAB+) (FIG. 1B) and 1 mM aniline and 1 mM N-phenyl-p-phenylenediamine (PPD) (labeled as PANI+) (FIG. 1C). Dashed lines represent the boundary of irradiation formed by the epifluorescence light source; Hoechst 33342 nuclear stain is shown. FIG. 1D shows that PDAB and PANI deposition were quantified by measuring the pixel brightness of cells in the transmitted light images and normalizing it to the average background of the cell-free parts of the image. Values represent mean±s.e.m., unpaired, two-tailed t-tests, ****, p<0.0001. FIGS. 1E-1F show merged confocal fluorescence and transmitted light (TL) images of fixed rat cortical neurons expressing miniSOG (miniSOG+ neurons) after 7 minutes of irradiation in the presence of 1 mM DAB (FIG. 1E) and 1 mM aniline and 1 mM PPD (FIG. 1F). FIGS. 1G-1H show corresponding images of neurons expressing only mCherry (miniSOG-neurons). FIGS. 1I-1J show insulating (FIG. 1I) and conductive (FIG. 1J) polymer deposition quantified as described in (FIG. 1D).

FIGS. 2A-2D provide schematics, images, and bar graphs showing optogenetic polymerization and assembly of electrically functional polymers in living neurons. FIG. 2A provides schematics of iterative optogenetic polymerization in living neurons. FIG. 2B provides representative confocal images showing primary miniSOG+ and miniSOG− neurons before and after PDAB (top) or PANI (bottom) optogenetic polymerization. The optogenetic polymerization was performed by immersing the neurons in Tyrode's solution containing 1 mM DAB or 0.5 mM aniline-PPD mixture at 1:1 molar ratio and being irradiated with blue light for 5-7 min (PDAB reaction) or 9-15 min (PANI reaction). FIG. 2C provides schematics illustrating the viability test of cells after miniSOG-catalyzed polymerization under different conditions by live/dead ratio assay. FIG. 2D provides statistical results of live/dead ratios calculated from the 9 groups. 4 replicates per group were performed. Values represent the ratio of live/dead cells, mean±s.e.m. Unpaired two-tailed t-tests; all p-values>0.05. Light intensities used for miniSOG-catalyzed polymerization are approximately 5 mW/mm².

FIGS. 3A-3D provide schematics, current plots, and bar graphs showing optically controlled and genetically targeted modulation of single-neuron excitability. FIG. 3A provides schematics of electrophysiological characterization on the cultured neurons. Top: whole-cell recordings were performed in the Tyrode's solution in the absence of monomers (Step 1), and then neurons were perfused with Tyrode's solution containing 1 mM DAB or 0.5 mM each aniline and PPD. Neurons were then irradiated with blue light (Step 2). After irradiation, monomer solutions were washed away by normal Tyrode's solution. During the entire process, neurons were maintained under whole-cell clamp mode for recording (Step 3). The setup for whole-cell recording includes an amplifier (AMP), a low pass filter (LPF) and an analogue-to-digital converter (ADC). Bottom: schematics of the recorded neuron showing the membrane properties measured by whole-cell patch-clamp before and after optogenetic polymerization. Vₚ is the pipette voltage; Rₚ is the pipette resistance; Cₚ is the pipette capacitance; R_{feedback} is the resistance of a feedback resistor; Rₘₑₘ is the membrane resistance; Cmem is the membrane capacitance; and Vₒᵤₜ is the output voltage. FIG. 3B provides representative current responses evoked by a 10 mV hyperpolarization step in voltage clamp before and after PDAB (top panel) or PANI polymerization (bottom panel). Cells were held at −70 mV and then hyperpolarized to −80 mV for 500 ms followed by −70 mV holding. The charge and discharge current waveforms were analyzed to extract the capacitance and resistance. FIGS. 3C-3D show comparison of membrane capacitance (FIG. 3C) and resistance (FIG. 3D) before and after PDAB or PANI polymerization (n=6 neurons for miniSOG−/PDAB, miniSOG+/PDAB, and miniSOG−/PANI groups; n=7 neurons for miniSOG+/PANI group). Bar graphs represent mean±s.e.m. Paired two tailed t-test. *, p<0.05; t-tests with non-significant p-values are omitted for clarity.

FIGS. 4A-4C provide schematics, current plots, and bar graphs showing current-injection-evoked spikes characterization before and after optogenetic polymerization of functional polymers in living neurons. Same polymerization procedures were applied on cultured primary miniSOG+ and miniSOG− cultured neurons as those in FIG. 2A. FIG. 4A shows representative traces evoked by stepwise tonic current injection (20 pA per step from −100 pA to 280 pA) before and after PDAB polymerization reaction (left). From top to bottom, action potentials were evoked at rheobase, rheobase+40 pA, and rheobase+80 pA in the current clamp mode, respectively. FIG. 4B shows representative traces evoked by stepwise tonic current injection (20 pA per step from −100 pA to 280 pA) before and after PANI polymerization (right). The traces were recorded under current clamp mode. Cells were held at −70 to −75 mV potential by injecting current. Stepwise tonic current (20 pA per step from −100 pA to 280 pA) were injected into cells to elicit action potentials and to determine rheobases. From top to bottom, action potentials were evoked at rheobase, rheobase+40 pA or rheobase+80 pA respectively. FIG. 4C shows comparison of spike numbers before and after PDAB or PANI polymerization at rheobase, rheobase+40 pA or rheobase+80 pA in current clamp (n=6 neurons in miniSOG−/PDAB+ group, n=6 neurons in miniSOG+/PDAB group, n=6 neurons in miniSOG−/PANI, n=7 neurons in miniSOG+/PANI group). All individual cells were maintained in the whole-cell patch-clamp configuration across pre-reaction and post-reaction time points for direct comparison. Bar graphs represent mean s.e.m. Paired two tailed t-test, *p<0.05, **p<0.01; t-tests with non-significant p-values are omitted for clarity.

FIGS. 5A-5K provide schematics and bar graphs showing stepwise modulation of single-neuron excitability. FIG. 5A shows schematics of electrophysiological recording on cultured neurons for change of membrane properties during stepwise polymerization reaction. FIGS. 5B-5C show change of membrane capacitance and resistance before and after progressively extended blue light irradiation (˜5 mW/mm², 5, 6, and 7 minutes) induced PDAB reaction. FIGS. 5D-5E show change of membrane capacitance and resistance before and after progressively extended blue light irradiation (˜5 mW/mm², 6, 7, and 8 minutes) induced PANI reaction. FIGS. 5F-5H show change of spike number at rheobase (FIG. 5F), rheobase+40 pA (FIG. 5G) and rheobase+80 pA (FIG. 5H) before and after progressively extended blue light irradiation (˜5 mW/mm², 5, 6, and 7 minutes) induced PDAB reaction. FIGS. 5I-5K show change of spike number at rheobase (FIG. 5I), rheobase+40 pA (FIG. 5J) and rheobase+80 pA (FIG. 5K) before and after progressively extended blue light irradiation (˜5 mW/mm², 6, 7, and 8 minutes) induced PANI reaction. All individual cells were maintained in the whole-cell patch-clamp configuration across pre-reaction and post-reaction time points for direct comparison. Bar graphs represent mean±s.e.m. Paired two tailed t-test, *p<0.05, **p<0.01. t-tests with non-significant p-values are omitted for clarity.

FIG. 6 provides a proposed molecular mechanism of miniSOG-catalyzed optogenetic polymerization. Irradiation of miniSOG (polypeptide-ribbon model) converts triplet oxygen (light-grey spheres) to its first excited, singlet state (dark-grey spheres). In the presence of reactive monomers (hexagons), singlet oxygen is rapidly quenched through a charge transfer complex, generating aminium radical cations, which subsequently polymerize and precipitate onto or within the membrane (brown aggregates) once a sufficient chain length is reached.

FIG. 7 provides a schematic showing comparison of cell toxicity among miniSOG− catalyzed optogenetic polymerization, chromophore assisted light inactivation, and photoablation. Top: In the technique discussed herein, singlet oxygen (dark-grey spheres) generated by miniSOG (polypeptide-ribbon model) upon irradiation quickly reacts with relatively high (1 mM) concentrations of quenching monomers (hexagons), coating the cell with functional polymers. Middle: In the previous example of miniSOG CALI targeting synaptic vesicle proteins, miniSOG is fused with VAMP2 or synaptophysin (dark-grey ovals). Singlet oxygen reacts with amino acid side chains in both the fused protein and its interactors, deactivating them, and preventing synaptic vesicle fusion. Bottom: In the previous example of miniSOG photoablation, miniSOG is targeted to the mitochondrial matrix, disrupting cellular respiration and ultimately leading to complete cell death.

FIGS. 8A-8H provide images and scatter-plots showing spatial specificity of PANI and PDAB optogenetic polymerization. FIGS. 8A-8D show from left to right: fluorescence images showing fluorescence of miniSOG (light-grey) and Hoechst 33342 (dark-grey) in fixed (FIGS. 8A-8B) and living (FIGS. 8C-8D) HEK cells prior to light exposure; TL images showing the cells before and after polymerization of PDAB (FIGS. 8A and 8C) and PANI (FIGS. 8B and 8D); Correlation between initial miniSOG fluorescence and darkening of cells in TL image after irradiation. Normalized fluorescence and TL brightness were calculated as described in FIGS. 1A-1J. R²=0.77, p<0.0001 (FIG. 8A); R²=0.58, p<0.0001 (FIG. 8B). R²=0.89, p<0.0001 (FIG. 8C); and R²=0.30, p<0.0001 (FIG. 8D). FIGS. 8E-8F show light-patterned optogenetic polymerization of DAB (FIG. 8E) and PANI (FIG. 8F) in living HEK293T cells. Images represent the boundary of the light-irradiated area from the objective as in FIGS. 1A-1J. FIG. 8G provides bar and dot plots showing the level of the polymerization, quantified as in FIG. 1D; unpaired, two-tailed t-tests, ** p<0.01, **** p<0.0001. FIG. 8H shows representative UV/Vis spectra of PDAB, PANI, and hydrochloric acid-doped PANI on miniSOG+HEK293T cells.

FIGS. 9A-9D provide images and bar graphs showing optogenetic polymerization of PANI in the presence and absence of oxygen. FIGS. 9A-9C show miniSOG-catalyzed polymerizations in neurons under different conditions. Neurons were fixed and treated with the monomer solution as described in the methods section. A thin stream of medical grade oxygen (FIG. 9A) or nitrogen (FIG. 9C) was bubbled through the monomer solution for 10-15 minutes immediately prior to polymerization, compared to monomer solution treated with neither gas (FIG. 9B). Fluorescence (miniSOG and mCherry) and TL images are shown at the boundary of irradiation after being exposed to the GFP filter set light source for 5 minutes). FIG. 9D shows relative darkening of cells in TL images, quantified as in FIGS. 1I-1J. *p<0.05; ***p<0.001, ****p<0.0001.

FIGS. 10A-10D provide images, bar graphs, and plots showing optogenetic polymerization of PANI with singlet oxygen or radical producing photosensitizers. FIG. 10A provides fluorescence images showing Hoechst, miniSOG, or the superoxide producing photosensitizing protein SuperNova Green, and mCherry. Images at the irradiation boundary were collected 10 minutes after irradiation. Blebbing of irradiated, SNG+ cells is noticeable, consistent with photoablation by the production of radical ROS in these cells. FIG. 10B shows quantification of polymerization as in FIG. 1D. FIG. 10C shows in vitro polymerization reactions were prepared in the dark, UV/vis spectra were acquired, then each well was irradiated with the same epifluorescence microscope light source using a standard Cy5 filter set for 30 sec, and the UV/vis spectra acquired again before significant insoluble precipitates formed. The wells were irradiated for another 30 seconds before collecting the image in panel, FIG. 10D. MB: 5 μM methylene blue, a singlet oxygen producing photosensitizer; PPD: 500 μM aniline dimer; SOD: 45 μg/mL superoxide dismutase; NaN₃: 5 mM sodium azide, a selective singlet oxygen quencher; VBBO: 5 μM Victoria Blue BO, a superoxide producing photosensitizer. ****p<0.0001.

FIG. 11 provides a Synthetic scheme for biotin-DAB.

FIGS. 12A-12I provides schematics, reaction scheme, and imaging of optogenetically patterned polymers at subcellular resolution. FIG. 12A shows schematics of methods for labeling polymers inside cells. Biotin-DAB is polymerized under the same condition as that used for cell viability and whole-cell patch-clamp measurement. Subsequently, the remaining monomers are washed out, and the biotin-containing PDAB is stained with a streptavidin-Alexa Fluor (AF) 647 conjugate. FIG. 12B shows structures of biotin-DAB and corresponding polymer. FIG. 12C shows merged confocal fluorescence images of fixed HEK293T cells co-expressing miniSOG and dTomato (light-grey), irradiated in the presence of 1 mM biotin-DAB, followed by staining with streptavidin-AF647 (dark-grey). FIG. 12D shows maximum intensity Z projection of streptavidin-AF647 staining in a polymerized and stained HEK293T cell. Dashed lines represent the re-slicing sections (right and bottom panels) along the x- and y-axis. FIGS. 12E-12G show confocal fluorescence images of AF647 and mCherry of representative miniSOG+(FIGS. 12E-12F) and miniSOG− (FIGS. 12G-12H) neurons after irradiation. The expression level was controlled to be similar to those used for cell viability and whole-cell patch-clamp characterizations: ˜3.2 mW/mm² irradiation of 475 nm light in the presence of 1 mM biotin-DAB followed by streptavidin-AF647 staining. FIG. 121, shows maximum intensity Z projection of streptavidin-AF647 staining in a polymerized and stained neuron. Dashed lines represent the re-slicing sections (right and bottom panels) along the x- and y-axis.

FIGS. 13A-13B provide schematics and images showing assessment of cell viability after polymerization. FIG. 13A shows schematics illustrating the optogenetic polymerization in living cells and staining for cell viability test. FIG. 13B shows representative images of acute cell viability tested immediately after polymerization reaction. 9 different groups were assessed. Light intensities used for polymerization are approximately 5 mW/mm².

FIGS. 14A-14C provide schematics, plots, and images showing controlling irradiation time and expression level of miniSOG to avoid neuronal hyperexcitability. FIG. 14A show schematics of the continuous whole-cell patch-clamp characterization on cultured primary neurons to optimize the reaction condition. The setup for whole-cell recording includes an amplifier (AMP), a low pass filter (LPF) and an analogue-to-digital converter (ADC). FIG. 14B shows representative traces evoked by phasic currents (500 pA, 10 ms, 5 Hz) injection after 5 min (short) and 10 min (long) irradiation. FIG. 14C shows neurons with the weak or high expression level of miniSOG are determined and selected based on the fluorescence intensity for the whole-cell patch-clamp characterization. Light intensities used for the optogenetic polymerization are approximately 5 mW/mm². Light intensities used for mCherry imaging are approximately 7 mW/mm².

FIGS. 15A-15D provide schematics and bar graphs showing membrane properties of neurons before and after light irradiation in the absence of DAB. FIG. 15A shows schematics of the continuous whole-cell patch-clamp characterization on cultured primary neurons before and after light irradiation for the characterization of membrane property and excitability. FIG. 15B shows membrane capacitance and membrane resistance measured by 10 mV hyperpolarization step in voltage clamp. The rheobase was determined by stepwise tonic current injection. Neurons were irradiated by blue light with the same intensity and duration as FIGS. 3A-3D (5 mW/mm², 7 minutes). FIG. 15C shows statistics of the stepwise tonic current-injection-evoked spikes at rheobase, rheobase+40 pA, and rheobase+80 pA in current clamp before and after light irradiation. FIG. 15D shows latency, half-width duration, and amplitude extracted from spikes evoked by phasic currents (500 pA, 10 ms, 5 Hz) injection. All individual cells were successfully maintained in the whole-cell patch-clamp configuration before and after the irradiation for direct comparison. Bar graphs represent mean s.e.m. dots with the same color within each group indicate the same neuron. Paired two tailed t-test. *p<0.05.

FIGS. 16A-16D provide schematics and bar graphs showing membrane properties of miniSOG− neurons before and after light irradiation in the absence of monomers. FIG. 16A shows schematics of the continuous whole-cell patch-clamp characterization on miniSOG− cultured primary neurons before and after light irradiation for the characterization of membrane property and excitability. FIG. 16B shows membrane capacitance and membrane resistance measured by 10 mV hyperpolarization step in voltage clamp. The rheobase was determined by stepwise tonic current injection. Neurons were irradiated by blue light with the same intensity and duration as FIGS. 3A-3D (5 mW/mm², 7 minutes). FIG. 16C shows statistics of the stepwise tonic current-injection-evoked spikes at rheobase, rheobase+40 pA, and rheobase+80 pA in current clamp before and after light irradiation. FIG. 16D shows latency, half-width duration, and amplitude extracted from spikes evoked by phasic currents (500 pA, 10 ms, 5 Hz) injection. All individual cells were successfully maintained in the whole-cell patch-clamp configuration before and after the irradiation for direct comparison. Bar graphs represent mean±s.e.m. dots with the same color within each group indicate the same neuron. Paired two tailed t-test. *p<0.05.

FIGS. 17A-17F provide schematics, plots, and bar graphs showing electrophysiological characterization of neurons before and after optogenetic polymerization of PDAB and PANI. FIG. 17A shows workflow of the continuous whole-cell patch-clamp characterization on cultured primary neurons before and after light irradiation for membrane kinetics measurement. FIG. 17B shows rheobase determined by stepwise tonic current injection. FIG. 17C shows representative traces evoked by phasic currents (500 pA, 10 ms, 5 Hz) injection. FIGS. 17D-17F show amplitude (FIG. 17D), latency (FIG. 17E) and half-width duration (FIG. 17F) extracted from spikes evoked by phasic currents (500 pA, 10 ms, 5 Hz) injection. All individual cells were maintained in the whole cell patch clamp configuration across pre-reaction and post-reaction time points for direct comparison. Bar graphs represent mean±s.e.m, dots with the same color within each group indicate the same neuron. Paired two tailed t-test. **p<0.01 versus group of before irradiation. Light intensities used for polymerization are approximately 5 mW/mm².

FIGS. 18A-18K provide schematics and bar graphs showing patch-clamp characterization and viability test 24 hours after optogenetic polymerization of PDAB and PANI. FIG. 18A shows a schematic of the workflow for measurements of viability and electrophysiological characterization of cultured primary neurons 24 hours after optogenetic polymerization of PDAB and PANI. Neurons were perfused with either Tyrode's solution (monomer-), Tyrode's solution containing 0.5 mM aniline and 0.5 mM PPD (PANI+) or 1.0 mM DAB (PDAB+) and irradiated with approximately 5 mW/mm² blue light that covers the whole plate. After irradiation, monomer or Tyrode's solutions were washed away and replaced with the original NbActiv4 medium and put back to the humid culture incubator with 5% CO₂ at 37° C. and incubated for another 24 hours. The neurons are then tested for viability with the same configurations as in FIG. 2C or whole-cell patch clamp characterizations. FIG. 18B shows statistical results of live/dead ratios. 6 groups per condition were performed. Values represent the ratio of live/dead cells. FIGS. 18C-18E show membrane capacitance (FIG. 18C) and resistance (FIG. 18D) measured by 10 mV hyperpolarization step in voltage clamp, and the rheobase (FIG. 18E) determined by stepwise tonic current injection. FIGS. 18F-18H show statistics of the stepwise tonic current-injection-evoked spikes at the rheobase (FIG. 18F), rheobase+40 pA (FIG. 18G) and rheobase+80 pA (FIG. 18H). FIGS. 18I-18K show latency (FIG. 18I), half-width duration (FIG. 18J) and amplitude (FIG. 18K) extracted from spikes evoked by phasic currents (500 pA, 10 ms, 5 Hz) injection. n=6 for each group. Bar graphs represent mean±s.e.m. Unpaired two-tailed t-test. *p<0.05.

FIGS. 19A-19K provide schematics and bar graphs showing patch-clamp characterization and viability test 3 days after optogenetic polymerization of PDAB and 15 PANI. FIG. 19A shows schematic of the workflow for measurements of viability and electrophysiological characterization of cultured primary neurons 3 days after optogenetic polymerization of PDAB and PANI with the same configurations as FIGS. 18A-18K. FIG. 19B shows statistical results of live/dead ratios. 6 groups per condition were performed. Values represent the ratio of live/dead cells. FIGS. 19C-19E show membrane capacitance (FIG. 19C) and resistance (FIG. 19D) measured by 10 mV hyperpolarization step in voltage clamp, and the rheobase (FIG. 19E) determined by stepwise tonic current injection. FIGS. 19F-19H show statistics of the stepwise tonic current-injection-evoked spikes at the rheobase (FIG. 19F), rheobase+40 pA (FIG. 19G) and rheobase+80 pA (FIG. 19H). FIGS. 19I-19K latency (FIG. 19I), half-width duration (FIG. 19J) and amplitude (FIG. 19K) extracted from spikes evoked by phasic currents (500 pA, 10 ms, 5 Hz) injection. n=6 for each group. Bar graphs represent mean±s.e.m. Unpaired two-tailed t-test. *p<0.05.

FIGS. 20A-20I provide schematics and bar graphs showing stepwise electrophysiological characterization of neurons before and after iterative optogenetic polymerization of PDAB and PANI. FIG. 20A shows schematics of electrophysiological recording on cultured neurons during the stepwise polymerization reaction. FIGS. 20B-20E show change of rheobase (FIG. 20B), amplitude (FIG. 20C), half-width duration (FIG. 20D), and latency (FIG. 20E) before and after multiple rounds of blue light irradiation (˜5 mW/mm², 5, 6, and 7 minutes) in the presence of DAB monomers. FIGS. 20F-20I show change of rheobase (FIG. 20F), amplitude (FIG. 20G), half-width duration (FIG. 20H), and latency (FIG. 20I) before and after multiple rounds of blue light irradiation in the presence of DAB monomers. All individual cells were maintained in the whole-cell configuration during the multiple rounds of irradiation for direct comparison. Bar graphs represent mean±s.e.m, dots with the same color within each group indicate the same neuron. Paired two tailed t-test. *p<0.05, **p<0.01.

### DETAILED DESCRIPTION OF THE INVENTION

The invention features compositions and methods for treating diseases or disorders associated with undesirable neuronal excitability (e.g., neurodegenerative disease, such as Parkinson's disease or Huntington's disease; or chronic pain, or epilepsy).

The disclosure is based, at least in part, on the discovery of viral vectors encoding a mini Singlet Oxygen Generator that when stimulated can induce the optogenetic assembly of conductive and insulating polymers that provide for the precise control of neuronal membrane capacitance and excitability.

### Compositions and Methods for Modifying Neuronal Excitability

Cell-type-specific neuronal intrinsic excitability changes are associated with many neurological disorders. Ionic conductivity and membrane capacitance are two foundational parameters that govern neuron excitability. Conventional optogenetic stimulation has proven to be a powerful tool to temporarily manipulate membrane ionic conductivity from genetically targeted components in intact biological systems. However, no analogous method exists for precisely manipulating cell membrane capacitance to enable long-lasting modulation of neuronal excitability with genetically targeted specificity. Genetically targetable polymerization and assembly of conductive and insulating polymers inside the cell can modulate cell membrane capacitance, but further development of this technique has been hindered by poor spatiotemporal control of the polymer deposition and cytotoxicity from the widely diffused reactive oxygen species. Herein, these issues are addressed by harnessing genetically targetable photosensitizer proteins to polymerize and assemble electrically functional polymers in neurons with precise spatiotemporal control enabled by light activation. Using whole-cell patch-clamp recordings, this optogenetic polymerization and assembly of electrically functional polymers was demonstrated, which can achieve stepwise increases or decreases of both the membrane capacitance and intrinsic excitability of neurons. Furthermore, cytotoxicity can be limited by controlling light exposure, demonstrating a promising new, optogenetically controlled, biosynthetic method for precisely modulating cell excitability.

As such, an engineered peroxidase was modified (Liu et al., Science. 80, (2020)) to be expressed in genetically specified neurons in brain tissues. The peroxidase can catalyze the oxidative polymerization of small molecule precursors into electrically functional (conductive or insulating) polymers at the plasma membrane in the presence of H₂O₂. Whole-cell patch-clamp showed that the in situ synthesized conductive/insulating polymers increased/decreased the membrane capacitance and reduced/elevated the excitability of polymer-coated neurons, respectively. This method shows promise to change the excitability of specific types of neurons in intact neural circuits. However, this peroxidase/H₂O₂-driven polymerization has the following limitations: (i) diffusion of H₂O₂ introduces acute toxicity to the neural systems and (ii) the H₂O₂-triggered polymerization cannot control the location and extent of in situ polymerization in neurons. These limitations prevent the further application of this technique to living cell membrane modulation with cellular and subcellular spatiotemporal resolution.

Here, these issues are addressed by developing an optically controlled, genetically targeted (optogenetic) polymerization and assembly of conductive and/or insulating polymers on the neuronal plasma membrane, which, akin to conventional optogenetic stimulations, not only precisely modulates the membrane capacitance in a light-controlled and stepwise manner, but also achieves cell-type-specific control over neuron excitability. To enable this optogenetic polymerization of electrically functional synthetic polymers, genetically targetable photosensitizer proteins were introduced to photopolymerize polyaniline (PANI) and poly(3,3′-diaminobenzidine) (PDAB) as conductive and insulating polymers throughout the cell, respectively. In some embodiments, the photosensitizer protein is miniSOG.

Whole-cell patch-clamp was used to characterize the electrophysiological properties of the neurons before and after the optogenetic polymerization, showing that the in situ synthesized conductive or insulating materials can increase or decrease the neuronal membrane capacitance and decrease or increase the intrinsic cellular excitability, respectively. Finally, it was shown that optogenetic polymerization can precisely control the location and density of polymers in cells by controlling the light intensity and exposure, thus enabling an iterative, stepwise increase/decrease of membrane capacitance.

### MiniSOG

miniSOG (for mini Singlet Oxygen Generator) is a 106 amino acid flavin-binding protein that generates singlet oxygen under exposure to blue light. It was originally developed by Shu and coworkers for correlative light and electron microscopy (CLEM) as it both fluoresces and catalyzes the photo-oxidation of diaminobenzidine (DAB), providing high-resolution images (Shu, X. et al. A genetically encoded tag for correlated light and electron microscopy of intact cells, tissues, and organisms. PLoS Biol. 9, e1001041 (2011)). miniSOG was engineered from the LOV2 (Light, Oxygen and Voltage) domain of Arabidopsis thaliana phototropin 2.

### Singlet Oxygen

Singlet oxygen (¹O₂) is a reactive oxygen species in which the electrons of the oxygen are in a singlet state (i.e., all of the electrons are spin paired). Singlet oxygen is more unstable and more reactive than ground state oxygen (³O₂), in which the electrons are in a triplet state (i.e., the molecule contains two spin unpaired electrons).

Singlet oxygen is commonly produced through the transfer of energy from a photosensitizer molecule to ground state oxygen.

Expression of Recombinant miniSOG

In one approach, a cell of interest (e.g., neuron, such as a motor neuron, sensory neuron, neuron of the central nervous system, or neuronal cell lines) is engineered to express a miniSOG polynucleotide whose expression renders the cell responsive to optical stimulation. Optical stimulation of such cells induces optogenetic polymerization and assembly of electrically functional polymers for modulation of neuronal excitability.

miniSOG may be constitutively expressed or its expression may be regulated by an inducible promoter or other control mechanism where conditions necessitate highly controlled regulation or timing of the expression of a miniSOG protein. For example, heterologous DNA encoding a miniSOG gene to be expressed is inserted in one or more pre-selected DNA sequences. This can be accomplished by homologous recombination or by viral integration into the host cell genome. The desired gene sequence can also be incorporated into a cell, particularly into its nucleus, using a plasmid expression vector and a nuclear localization sequence. Methods for directing polynucleotides to the nucleus have been described in the art. The genetic material can be introduced using promoters that will allow for the gene of interest to be positively or negatively induced using certain chemicals/drugs, to be eliminated following administration of a given drug/chemical, or can be tagged to allow induction by chemicals, or expression in specific cell compartments.

A variety of vectors can be used to introduce a miniSOG polynucleotide to a cell of interest. In one embodiment, transducing viral (e.g., retroviral, adenoviral, lentiviral and adeno-associated viral) vectors can be used to introduce a miniSOG to a cell, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a miniSOG nucleic acid molecule, can be cloned into a retroviral vector and expression can be driven from a neuronal promoter, a CAG promoter, synapsin promoter, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Calcium phosphate transfection can be used to introduce plasmid DNA containing a miniSOG polynucleotide into cells and is a standard method of DNA transfer to those of skill in the art. DEAE-dextran transfection, which is also known to those of skill in the art, may be preferred over calcium phosphate transfection where transient transfection is desired, as it is often more efficient. Since the cells of the present invention are isolated cells, microinjection can be particularly effective for transferring genetic material into the cells. This method is advantageous because it provides delivery of the desired genetic material directly to the nucleus, avoiding both cytoplasmic and lysosomal degradation of the injected polynucleotide. Cells can also be genetically modified using electroporation.

Liposomal delivery of DNA or RNA to genetically modify the cells can be performed using cationic liposomes, which form a stable complex with the polynucleotide. For stabilization of the liposome complex, dioleoyl phosphatidylethanolamine (DOPE) or dioleoyl phosphatidylcholine (DOPQ) can be added. Commercially available reagents for liposomal transfer include Lipofectin (Life Technologies). Lipofectin, for example, is a mixture of the cationic lipid N-[1-(2, 3-dioleyloxy)propyl]-N—N—N— trimethyl ammonia chloride and DOPE. Liposomes can carry larger pieces of DNA, can generally protect the polynucleotide from degradation, and can be targeted to specific cells or tissues. Cationic lipid-mediated gene transfer efficiency can be enhanced by incorporating purified viral or cellular envelope components, such as the purified G glycoprotein of the vesicular stomatitis virus envelope (VSV-G). Gene transfer techniques which have been shown effective for delivery of DNA into primary and established mammalian cell lines using lipopolyamine-coated DNA can be used to introduce target DNA into the de-differentiated cells or reprogrammed cells described herein.

Naked plasmid DNA can be injected directly into a tissue comprising cells of interest. Microprojectile gene transfer can also be used to transfer genes into cells either in vitro or in vivo. The basic procedure for microprojectile gene transfer was described by J. Wolff in Gene Therapeutics (1994), page 195. Similarly, microparticle injection techniques have been described previously, and methods are known to those of skill in the art. Signal peptides can be also attached to plasmid DNA to direct the DNA to the nucleus for more efficient expression.

Viral vectors are used to genetically alter cells of the present invention and their progeny. Viral vectors are used, as are the physical methods previously described, to deliver one or more polynucleotide sequences encoding miniSOG, for example, into the cells. Viral vectors and methods for using them to deliver DNA to cells are well known to those of skill in the art. Examples of viral vectors that can be used to genetically alter the cells of the present invention include, but are not limited to, adenoviral vectors, adeno-associated viral vectors, retroviral vectors (including lentiviral vectors), alphaviral vectors (e. g., Sindbis vectors), and herpes virus vectors.

### Adeno-Associated Virus (AAV)

In embodiments, the miniSOG is delivered by an AAV. AAV is a small (25 nm), nonenveloped virus that contains a linear single-stranded DNA genome packaged into the viral capsid. It belongs to the family Parvoviridae and is of the genus Dependovirus, because productive infection by AAV occurs only in the presence of either an adenovirus or herpesvirus helper virus. In the absence of helper virus, AAV (serotype 2) can establish latency after transduction into a cell by specific but rare integration into chromosome 19q13.4. Accordingly, AAV is the only mammalian DNA virus known to be capable of site-specific integration. (Daya, S. and Berns, K I., 2008, Clin. Microbiol. Rev., 21(4):583-593).

There are two stages to the AAV life cycle after successful infection: a lytic stage and a lysogenic stage. In the presence of adenovirus or herpesvirus helper virus, the lytic stage persists. During this period, AAV undergoes productive infection characterized by genome replication, viral gene expression, and virion production. The adenoviral genes that provide helper functions for AAV gene expression include E1a, E1b, E2a, E4, and VA RNA. While adenovirus and herpesvirus provide different sets of genes for helper function, they both regulate cellular gene expression and provide a permissive intracellular milieu for a productive AAV infection. Herpesvirus aids in AAV gene expression by providing viral DNA polymerase and helicase as well as the early functions necessary for HSV transcription.

rAAV as a Vector for Gene Delivery and Therapeutic Treatment

AAVs are well suited for use as vectors and vehicles for gene transfer to the nervous system. AAVs provide safe, long-term expression in the nervous system. Most of the foregoing applications rely on local AAV injections into the adult brain to bypass the blood-brain barrier (BBB) and to temporally and spatially restrict transgene expression.

AAV vectors have been highly successful in fulfilling all of the features desired for a delivery vehicle, such as the ability to attach to and enter the target cell, successful transfer to the nucleus, the ability to be expressed in the nucleus for a sustained period of time, and a general lack of pathogenicity and toxicity. Recombinant AAV (rAAV) is advantageous as a delivery vector, particularly for delivery to neurons in brain tissue, as it is focally injectable; it exhibits stable expression over time; and it is both non-pathogenic and non-integrative into the genome of the cell into which it is transduced. Twelve human serotypes of AAV (AAV serotype 1 (AAV-1) to AAV-12) and more than 100 serotypes from nonhuman primates have been reported to date. (Daya, S. and Berns, K. I., 2008, Clin. Microbiol. Rev., 21(4):583-593). In addition, rAAV has been approved by the FDA for use as a vector in at least 38 protocols for a number of different human clinical trials. AAV's lack of pathogenicity, persistence and its many available serotypes have increased the potential of the virus as a delivery vehicle for a gene therapy application in accordance with the described compositions and methods.

rAAV vectors have been constructed that do not encode the replication (Rep) proteins and that lack the cis-active, 38 base pair integration efficiency element (IEE), which is required for frequent site-specific integration. The inverted terminal repeats (ITRs) are retained because they are the cis signals required for packaging. Thus, current recombinant AAV (rAAV) vectors persist primarily as extrachromosomal elements.

Recombinant AAV (rAAV) vectors for gene therapy have been based mostly on the AAV-2 serotype. AAV-2-based rAAV vectors can transduce muscle, liver, brain, retina, and lungs, requiring several weeks for optimal expression. The efficiency of rAAV transduction is dependent on the efficiency at each step of AAV infection, i.e., virus binding, entry, trafficking, nuclear entry, uncoating, and second-strand synthesis.

Several novel AAV vector technologies have been developed to either increase the genome capacity for AAV or enhance gene expression. Trans-splicing AAV vectors have been used to increase the capacity of the vector for harboring heterologous polynucleotides by taking advantage of AAV's ability to form head-to-tail concatemers via recombination in the ITRs. In this approach, the transgene cassette is split between two rAAV vectors containing adequately placed splice donor and acceptor sites. Transcription from recombined AAV molecules, followed by the correct splicing of the mRNA transcript, results in a functional gene product. While somewhat less efficient than rAAV vectors, trans-splicing AAV vectors permit delivery of therapeutic genes up to 9 kb in size and have been successfully used for gene expression in the retina, lung and muscle.

rAAV polynucleotides may include additional elements, for example, a sequence encoding a reporter or a detectable marker, such as a fluorescent protein, or an element such as a Woodchuck Hepatitis Virus Posttrascriptional Regulatory Element (WPRE), which may increase RNA stability and protein yield. An rAAV polynucleotide may also comprise a promoter to drive transcription of one or more polynucleotides (genes) which are inserted between inverted terminal repeats (ITRs). A polyadenylation signal, such as bovine growth hormone polyadenylation signal and/or SV40 polyomavirus simian virus 40 polyadenylation signal, may be included as elements in the rAAV polynucleotide. The rAAV polynucleotide can comprise a minimal promoter, e.g., a human beta-globin minimal promoter (phog) and a chimeric intron sequence (Hermeming et al., 2004, J Virol Methods, 122(1):73-77). Without wishing to be bound by theory, ITRs may aid in concatamer formation in the nucleus after the single-stranded, AAV vector DNA is converted into double stranded (ds) DNA by host cell DNA polymerase complexes. Thus, the administration of the described rAAVs may form episomal concatemers in the nucleus of neuronal cells into which they are transduced. In non-dividing cells, concatemers may remain intact in these cells for the lifetime of the neurons. Advantageously, integration of rAAV polynucleotides into host chromosomes is likely to be negligible or absent and will not alter or affect the expression or regulation of any other human gene.

Recombinant AAV vectors can be made using standard and practiced techniques in the art and employing commercially available reagents. It will be appreciated by the skilled practitioner that rAAV vectors that been used in several clinical trials that have yielded promising results. By way of example, rAAV based therapy received marketing approval by the European Union in 2012, as reported by Kotterman, M. A. et al., 2014, Nat. Rev. Genet., 15:445-451. In some embodiments, plasmid vectors may encode all or some of the well-known replication (rep), capsid (cap) and adeno-helper components. The rep component comprises four overlapping genes encoding Rep proteins required for the AAV life cycle (e.g., Rep78, Rep68, Rep52 and Rep40). The cap component comprises overlapping nucleotide sequences of capsid proteins VP1, VP2 and VP3, which interact together to form a capsid of an icosahedral symmetry. A second plasmid that encodes helper components and provides helper function for the AAV vector may also be co-transfected into cells. The helper components comprise the adenoviral genes E2A, E4orf6, and VA RNAs for viral replication.

In an embodiment, a method of making rAAVs for the products, compositions, and uses described herein involves culturing cells that comprise an rAAV polynucleotide expression vector as described; culturing the cells to allow for expression of the polynucleotides to produce the rAAVs within the cell, and separating or isolating the rAAVs from cells in the cell culture and/or from the cell culture medium. Such methods are known and practiced by those having skill in the art. The rAAVs can be purified from the cells and cell culture medium to any desired degree of purity using conventional techniques.

### Cell-Specific AAV Capsids

The rational design of AAV vectors that display selective tissue/organ targeting has broadened the applications of AAV as vector/vehicle for gene therapy. Both direct and indirect targeting approaches have been used to enhance AAV vector cell targeting specificity and retargeting. By way of example, in direct targeting, AAV vector targeting to certain cell types is mediated by small peptides or ligands that have been directly inserted into the viral capsid sequence. This approach has been successfully employed to target endothelial cells. Direct targeting requires detailed knowledge of the capsid structure such that peptides or ligands are positioned at sites that are exposed to the capsid surface; the insertion does not significantly affect capsid structure and assembly; and the native tropism is ablated to maximize targeting to a specific cell type. In indirect targeting, AAV vector targeting is mediated by an associating molecule that interacts with both the viral surface and the specific cell surface receptor. Such associating molecules for AAV vectors may include bispecific antibodies and biotin. The advantages of indirect targeting are that different adaptors can be coupled to the capsid without resulting in significant changes in the capsid structure, and the native tropism can be easily ablated. A disadvantage of using adaptors for targeting involves a potential for decreased stability of the capsid-adaptor complex in vivo.

In addition, AAV vectors may be produced that comprise capsids that allow for the increased transduction of cells and gene transfer to the central nervous system and the brain via the vasculature. (Chan, K. Y. et al., 2017, Nat. Neurosci., 20(8):1172-1179). Such vectors facilitate robust transduction of neuronal. When used with enhancers and cell-type specific promoters, such AAVs provide targeted gene expression in neuronal cells of the nervous system.

For applications that do not require high expression levels per cell, the amount of virus used, i.e., the viral dose, could be lowered. Lowering the viral load used for systemic gene delivery can reduce cost and production burden and minimize a potential risk for adverse reactions to viral components.

To achieve enhanced therapy or treatment, the dose of AAV vector that is required for a therapeutic response may be reduced, e.g., by using certain AAV serotypes. Alternatively, the surface of the AAV vector capsid may be altered to include specific ligands for attachment to target tissues and cells as described above. Another approach takes into consideration the trafficking of the virus particle from the endocytoplasmic vesicle to the nucleus. (Zhao, W. et al., 2007, Gene Ther., 14:545-550; Daya, S. and Berns, K. I., 2008, Clin. Microbiol. Rev., 21(4):583-593). Typically, the virus particle-to-infectivity ratio of AAV vector preparations ranges from 10:1 to 100:1. The high ratios reflect incomplete or empty vector particles, as well as trafficking from the endocytoplasmic vesicle to the nucleus. During trafficking, the vector particle may become ubiquitinated and directed to a proteasome for degradation, rather than to the nucleus where the transgene may be expressed. It was found that ubiquitination and direction to the proteasome require phosphorylation of tyrosine residues on the surface of the AAV vector capsid. When the seven tyrosine residues on the surface of the AAV-2 capsid were replaced phenylalanine residues, the multiplicity of infection (MOI) required for the detection of transgene expression was greatly reduced both in cell culture and in several mouse models of transduction of cells in the liver and eye. Consequently, the ability to increase transgene expression to therapeutic levels in the treatment of diseases may be enhanced.

One or more treatment approaches to gain control over seizures are embraced by the therapeutic products, compositions and methods described herein involving state-of-the-art gene therapy or pharmaco-genetic approaches. Such approaches may likely lead to the development of a clinically relevant therapies to alleviate the seizure symptoms of epilepsy.

For direct delivery to the brain, AAV vectors may be administered by open neurosurgical procedure or by focal injection in order to bypass the blood-brain barrier, to temporally and spatially restrict transgene expression, and to target specific areas of the brain.

Systemic AAV delivery (by intravenous injection) provides a non-invasive alternative for broad gene delivery to the nervous system. Several groups have developed AAV capsids that enhance gene transfer to the CNS and certain tissues and cell populations after intravenous delivery. By way of example, AAV-AS capsid18 utilizes a polyalanine N-terminal extension to the AAV9.4719 VP2 capsid protein to provide higher neuronal transduction, particularly in the striatum. The AAV-BR1 capsid20, based on AAV2, may be useful for more efficient and selective transduction of brain endothelial cells. Another AAV capsid, AAV-PHP.eB, comprises a capsid that transduces the majority of neurons and astrocytes across many regions of the adult mouse brain and spinal cord after intravenous injection.

Other modes of AAV vector administration may include lipid-mediated vector delivery, hydrodynamic delivery, and a gene gun.

### Promoters

Promoters useful in expressing a miniSOG polynucleotide in a cell (e.g., neuronal cell) are known in the art and are described herein. In one embodiment, the promoter is a CAG promoter constructed from the (C) Cytomegalovirus (CMV) early enhancer element; (A) the promoter, the first exon and the first intron of chicken beta-Actin gene, and (G) the splice acceptor of the rabbit beta-Globin gene. The CAG promoter is known in the art and described, for example, by Miyazaki et al., Gene. 79 (2): 269-77, 1989 and Niwa et al., Gene. 108 (2): 193-9, 1991, each of which is incorporated herein by reference in their entirety.

In another embodiment, the promoter is a human synapsin promoter. The human synapsin 1 gene promoter confers highly specific long-term expression in the brain. Synapsin promoters are described, for example, by Kugler et al., Gene Therapy 10:337-347, 2003, by Jackson et al., Front. Mol. Neurosci., 4 Nov. 2016, Sec. Methods and Model Organisms.

Other promoters expressed in neurons are known in the art.

### Treatment of Disorders Characterized by Undesirable Activity

The virus vectors comprising miniSOG and compositions thereof (comprising miniSOG to photopolymerize polyaniline (PANI) and poly(3,3′-diaminobenzidine) (PDAB)) as described herein may be used in the treatment of neurological and neurodegenerative diseases and disorders, particularly, for the treatment of epilepsy. A characteristic that distinguishes categories of seizures is whether the seizure activity is partial (e.g., focal) or generalized. In an embodiment, virus vectors and compositions thereof as described herein are used to treat partial and/or generalized seizures. Partial seizures are typically considered to be those in which the seizure activity is restricted to discrete areas of the cerebral cortex. As will be appreciated by the skilled practitioner, a seizure is characterized as a simple-partial seizure if consciousness is fully preserved during the course of the seizure. If consciousness is impaired, then the seizure is characterized as a complex-partial seizure. Complex-partial seizures also include those that initiate as partial seizures and subsequently extend through the cortex; as such, these types of seizures are considered to be partial seizures with secondary generalization.

Generalized seizures encompass distant regions of the brain simultaneously in a bilaterally symmetric manner and can include sudden, brief lapses of consciousness, such as in the case of absence or petit mal seizures, without loss of postural control. Atypical absence seizures usually include a longer period of lapse of consciousness and more gradual onset and termination. Generalized tonic-clonic or grand mal seizures, considered as the main type of generalized seizures, typically have an abrupt onset without warning. The initial phase of the seizure usually involves tonic contraction of muscles, impaired respiration, a marked enhancement of sympathetic tone leading to increased heart rate, blood pressure and pupil size. After approximately 10-20 seconds, the tonic phase of the seizure typically evolves into a clonic phase, which is produced by periods of muscle relaxation superimposed on the tonic muscle contraction. The periods of relaxation progressively increase until the end of the ictal phase, which usually lasts no more than one minute. The postictal phase is characterized by unresponsiveness, muscular flaccidity, and excessive salivation that can cause stridorous breathing and partial airway obstruction.

Atonic seizures are characterized by sudden loss of postural muscle tone lasting approximately 1-2 seconds. While consciousness is briefly impaired, there is usually no postictal confusion. Myoclonic seizures are characterized by a sudden and brief muscle contraction that may involve one part of the body or the entire body. Without limitation, the rAAV products, compositions and methods of use thereof as described herein embrace the prophylactic and/or therapeutic treatment of the above-described seizures, including those associated with epilepsy. In an embodiment, the rAAV products, compositions and methods of use thereof as described herein are used for the prophylactic and/or therapeutic treatment of epilepsy.

### Targeted Cell Types

miniSOG can be expressed in virtually any eukaryotic or prokaryotic cell of interest. In one embodiment, the cell is a neuronal cell type that requires modulation of its excitability. In another embodiment, the cell is a human neuron (e.g., motor neuron, sensory neuron, neuron of the central nervous system, and neuronal cell line).

### Methods of Stimulating a Neural Cell

The methods provided herein are, inter alia, useful for the stimulation of the neuronal cells. In particular, irradiation and/or light stimulation induces photopolymerization of polyaniline (PANI) and/or poly(3,3′-diaminobenzidine) (PDAB) as conductive and insulating polymers. Expression of miniSOG in a cell and subsequent photostimulation induces polymerization of PANI and/or PDAB, which alters the excitability of the cell.

The term “neural cell” as provided herein refers to a cell of the brain or nervous system. Non-limiting examples of neural cells include neurons, glia cells, astrocytes, oligodendrocytes and microglia cells. Where a neural cell is stimulated, a function or activity (e.g., excitability) of the neural cell is modulated by modulating, for example, the expression or activity of a given gene or protein (e.g., miniSOG) within said neural cell. The change in expression or activity may be 10%, 20%, 30%, 40%, 50%, 60%, 70%, 80%, 90% or more in comparison to a control (e.g., unstimulated cell). In certain instances, expression or activity is 1.5-fold, 2-fold, 3-fold, 4-fold, 5-fold, 10-fold or higher than the expression or activity in the absence of stimulation. In certain instances, expression or activity is 1.5-fold, 2-fold, 3-fold, 4-fold, 5-fold, 10-fold or lower than the expression or activity in the absence of stimulation. The neural cell may be stimulated by irradiating the neural cell.

The term “applying” as provided herein is used in accordance with its plain ordinary meaning and includes the meaning of the terms contacting, introducing and exposing. In embodiments, the neural cell forms part of an organism. In embodiments, the organism is a bacterial cell or mammalian cell (e.g., human, murine, bovine, feline, canine).

Stimulation is achieved by exciting the cell using energy of various wavelengths. In particular embodiments, light is used. In one embodiment, blue light irradiation is used. In other embodiments, light of other colors or of multiple colors is used. In particular embodiments, a neuron is irradiated with 300, 350, 375, 400, 425, 450, 475, 500, 525, 550 nm. In embodiments, the irradiation continues for 1-20 minutes (e.g., 1, 2, 3, 4, 5, 6, 7, 8, 9, 10, 11, 12, 13, 14, 15 minutes).

### Methods of Treatment

In another aspect, a method of treating a disease or disorder characterized by undesirable neuronal activity (e.g., neurodegenerative disease, such as Parkinson's disease or Huntington's disease; or chronic pain, or epilepsy) in a subject in need thereof is provided. The method includes (i) administering to a subject a therapeutically effective amount of a vector comprising recombinant nucleic acid encoding miniSOG, as well as DAB and/or polyaniline. In step (ii) light stimulation (irradiation) is applied to a cell of the subject, resulting in optically controlled, genetically targeted (optogenetic) polymerization and assembly of conductive and/or insulating polymers on the neuronal plasma membrane. This assembly provides for a change in neuronal excitability. In one embodiment, the methods described herein treat a neurological disease by altering neural activity in the subject. In embodiments, the disease is a neurodegenerative disease, such as Parkinson's disease or Huntington's disease; or chronic pain, epilepsy, or another disease associated with an undesirable alteration in neuronal excitability.

### Kits

The invention provides kits for preventing or treating diseases and disorders characterized by undesirable neuronal activity, including neurodegenerative disorders, such as Parkinson's disease or Huntington's disease, as well as chronic pain, seizures and/or epilepsy. In one embodiment, the kit provides a therapeutic or prophylactic composition containing an effective amount of a rAAV vector or viral particle as described herein, which comprises a promoter (e.g., CAG) or neuronal specific promoter (e.g., human synapsis) that drives expression of miniSOG. In addition to the vector, the kit further comprises polyaniline (PANI) and poly(3,3′-diaminobenzidine) (PDAB) that are photopolymerized to form conductive and insulating polymers throughout the cell, respectively. In some embodiments, the kit comprises a sterile container which contains the therapeutic or prophylactic composition; such containers can be boxes, ampoules, bottles, vials, tubes, bags, pouches, blister-packs, or other suitable container forms known in the art. The containers can be made of plastic, glass, laminated paper, metal foil, or other materials suitable for holding medicaments.

A composition comprising an rAAV vector comprising at least a miniSOG polynucleotide sequence as described herein is provided together with instructions for administering the composition to a subject having or at risk of developing neurodegenerative disorders, such as Parkinson's disease or Huntington's disease, as well as chronic pain, seizures and/or epilepsy. The instructions will generally include information about the use of the composition for the treatment or prevention of the disease or disorder. In other embodiments, the instructions include at least one of the following: description of the therapeutic agent (rAAV comprising miniSOG polynucleotide sequence, PDAB, PANI etc.); dosage schedule and administration for treatment or prevention of the disease or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Optogenetic Polymerization of Poly(Diaminobenzidine) and Polyaniline in Living Cells

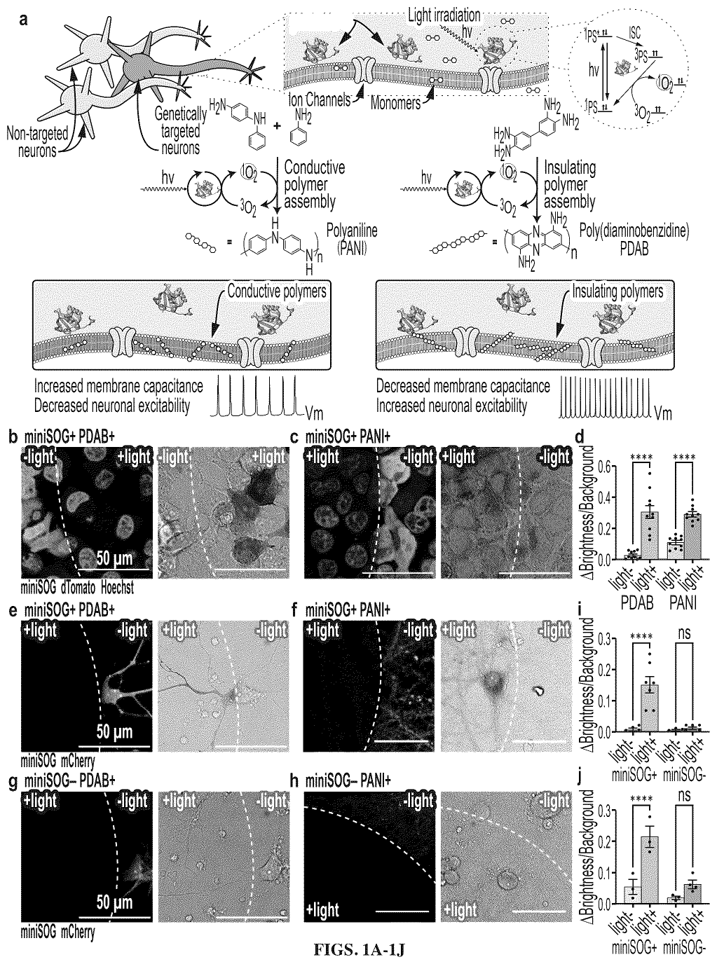

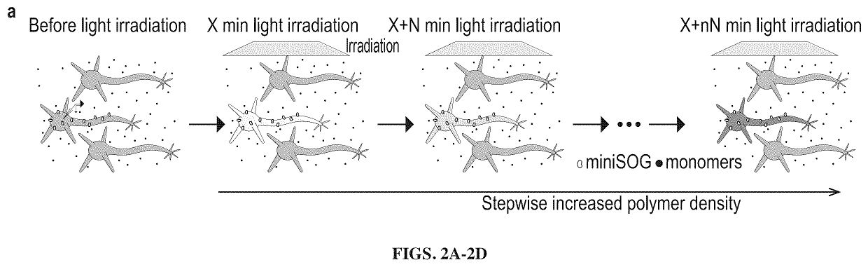

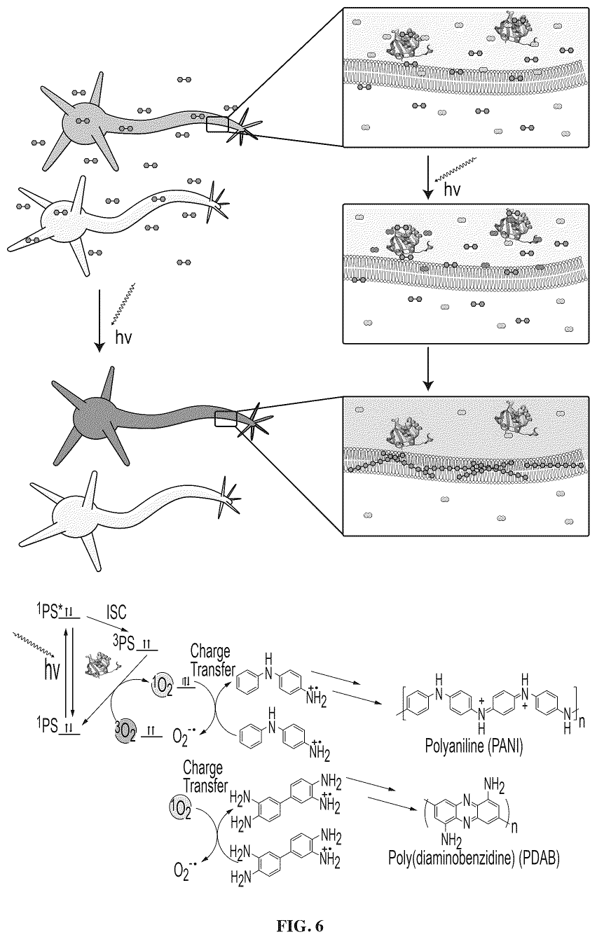

The photosensitizing protein mini singlet oxygen generator (miniSOG) was selected to facilitate optical control of poly(diaminobenzidine) (PDAB) and polyaniline (PANI) assembly on or within the cell membrane (FIG. 1A). Compared to other genetically targetable photosensitizers such as KillerRed and its derivatives, miniSOG produces more singlet oxygen relative to other reactive oxygen species (ROS), resulting in its ability to photopolymerize DAB with nanometer-level spatial resolution. In addition, it was hypothesized that this singlet oxygen generation could also synthesize the conductive polymer PANI inside cells (FIG. 6). Successful synthesis of PANI has been accomplished utilizing a mixture of aniline and the aniline dimer N-phenyl-p-phenylenediamine (PPD) photosensitized by the singlet oxygen generator Ru(bpy)₃²⁺ in aqueous solution. It was hypothesized that photosensitized PANI polymerization was feasible. PPD (unlike monomeric aniline) has been shown to be an effective singlet oxygen quencher, and related p-phenylenediamine derivatives have been shown to react with singlet oxygen via a charge transfer in aqueous solution, generating the radical cations necessary for aniline polymerization.

Furthermore, it was hypothesized that the short lifetime of singlet oxygen in the aqueous solution would lead to only a small (sub-micrometer scale) region of the cell near the expressed miniSOG exposed to the oxidant, which would allow the toxic effects of miniSOG to be spatially confined. In contrast to chromophore-assisted light inactivation (CALI) studies in which miniSOG protein fusions have been employed to perturb the function of specific protein targets under high light exposure (Lin et al., Neuron. 79, 241-253 (2013); Souslova et al., J Biophotonics. 10, 338-352 (2017)), it was hypothesized that carefully controlling light exposure and sparsely expressing miniSOG (FIGS. 1A and 7) can avoid both diffusive cytotoxicity and the specific inactivation of membrane proteins commonly targeted for CALI. In addition, quenching of singlet oxygen by the introduced DAB or PPD could also potentially limit singlet oxygen exposure.

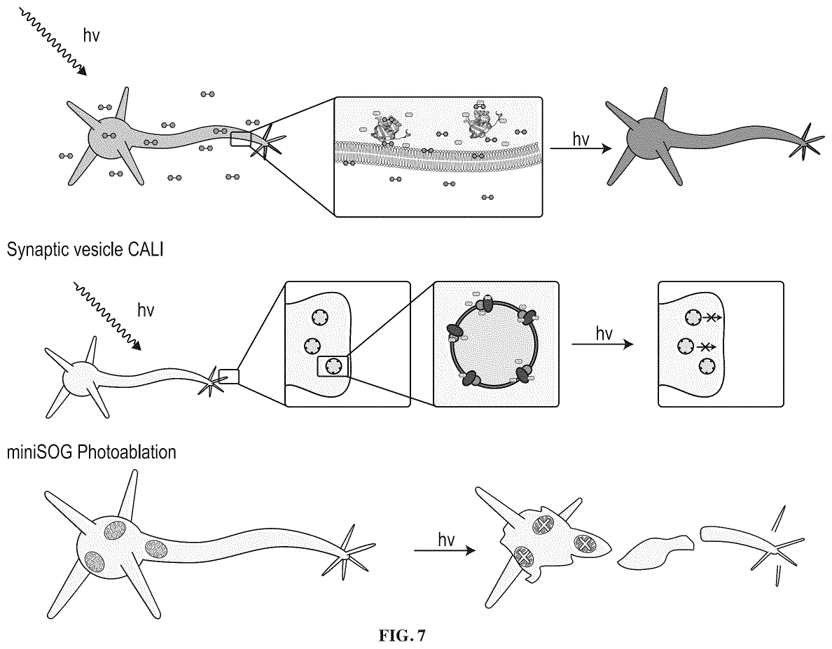

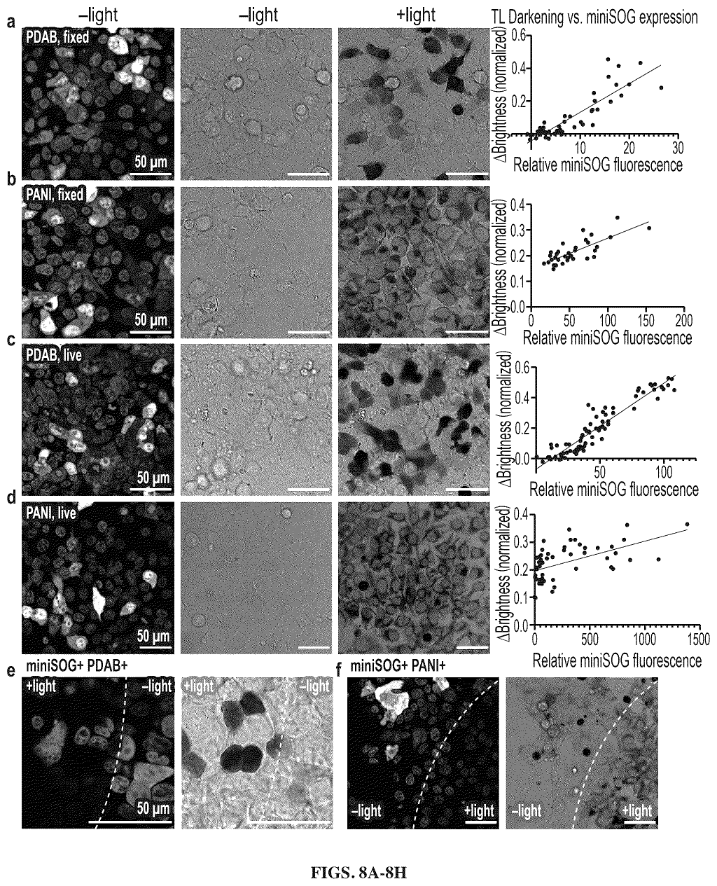

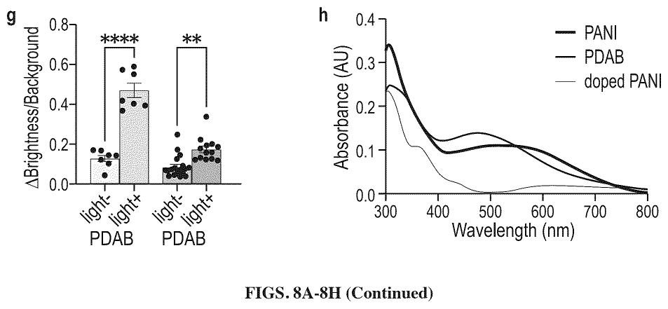

To assess the ability of miniSOG to polymerize DAB and PPD in living cells, miniSOG, joined with dTomato was expressed by a T2A ribosomal skip sequence, in the cytosol of HEK293T cells under the control of the CAG promoter. Irradiating fixed cells expressing this miniSOG construct (termed as miniSOG+ cells) with a standard GFP filter set in the presence of 1 mM DAB produced a dark brown precipitate in transmitted light (TL) images where miniSOG fluorescence was observed (FIGS. 1B and 1D). The darkening due to PDAB assembly correlated well with miniSOG expression, with non-transfected cells showing essentially no polymerization (FIG. 8A). Similarly, when fixed miniSOG+ cells were irradiated in a mixture of aniline and PPD, a lighter purple-gray precipitate (indicative of PANI) formed in only the irradiated cells (FIGS. 1C and 1D). Compared with PDAB, PANI polymerization also occurred primarily in transfected cells, but some nonspecific polymerization was observed in non-transfected cells, potentially resulting from non-sensitized photooxidation of PPD. An increased monomer concentration (1 mM each of aniline and PPD) was required to achieve a similar level of polymerization as characterized by the darkening of the reacted cells (FIG. 8B). Similar results were obtained in living cells for both polymer types, albeit with higher nonspecific background polymerization in the case of PANI (FIGS. 8C-8G). Notably, a clear boundary between polymerized and non-polymerized regions of cells was visible for PDAB (FIG. 8E), although a small amount of polymerization is observed outside the region exposed to the highest light intensity as a result of the non-coherent light source used. This demonstrated that a subcellular-level of spatially specific polymerization is achievable by optogenetic polymerization in living cells.

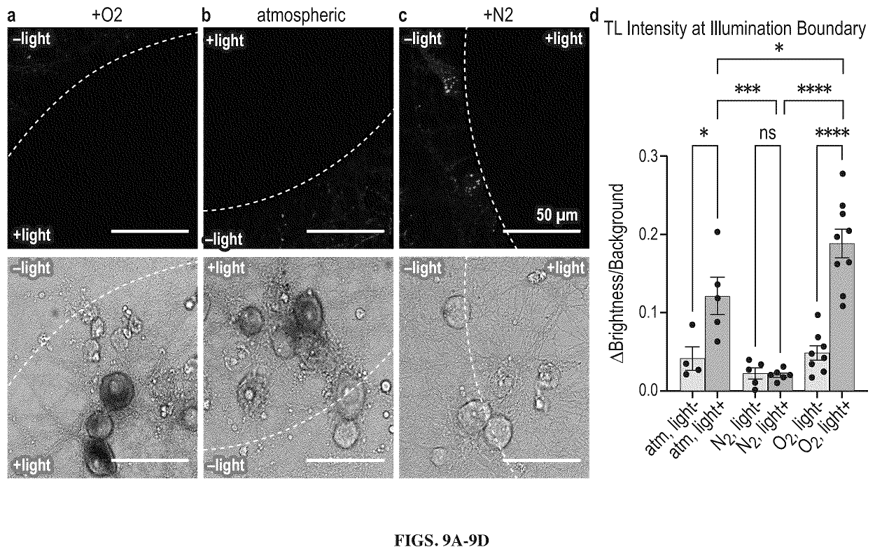

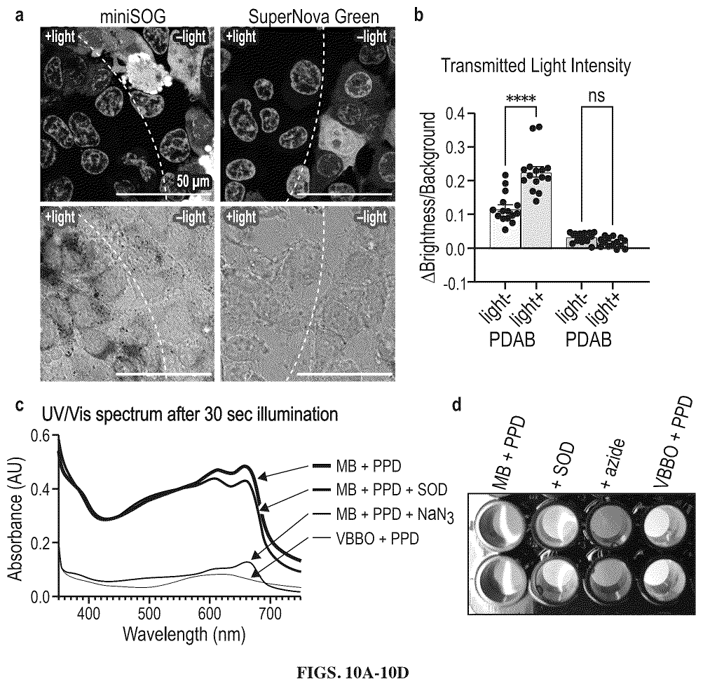

Ultraviolet-visible (UV/vis) spectroscopy was used to characterize the in situ optogenetically polymerized PANI and PDAB. The polymerized PANI displayed features consistent with chemically synthesized, undoped PANI, while PDAB exhibited a broad absorbance in the visible range consistent with literature reports (FIG. 8H). Notably, the PANI deposited on cells exhibited a red-shift consistent with chemically synthesized PANI upon acid-doping treatment with hydrogen chloride (FIG. 8H), indicating similar extended, conjugated structures for the miniSOG-catalyzed PANI formed on the cells. Consistent with the proposed mechanism, miniSOG-catalyzed PANI polymerization appeared to be primarily dependent on singlet oxygen. Specifically, deoxygenated monomer solutions formed little to no detectable PANI precipitate (FIGS. 9A-9D), as did photosensitizers primarily producing ROS other than singlet oxygen (FIGS. 10A-10D), demonstrating the importance of a singlet oxygen-producing photosensitizer for in situ PANI polymerization.

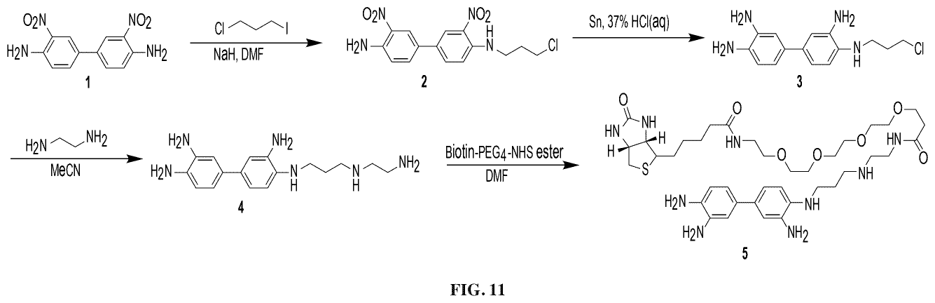

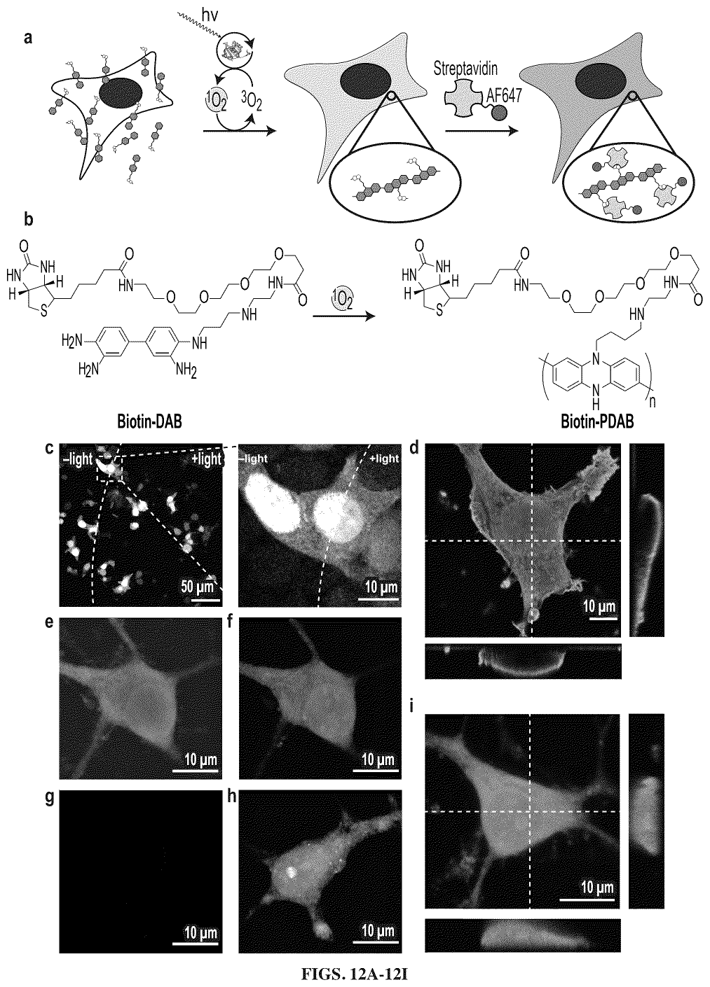

Because transmitted light darkening and electron microscopy both require a high amount of PDAB deposition for visualization, a more sensitive and direct method was devised for assessing the location of the deposited PDAB by fluorescently labeling the deposited polymer. This could not be achieved by directly tethering the DAB monomer to fluorophores, mainly due to the instability of most fluorophores toward singlet oxygen. Here, instead, the DAB monomer was tethered to biotin via a PEG linker (FIGS. 11 and 12A-12B). When miniSOG+ HEK293T cells were illuminated as above in the presence of 1 mM of the biotin-DAB conjugate, followed by staining with a streptavidin-Alexa Fluor™ 647 (AF647) conjugate, the AF647 signal was primarily observed in the darkened, light-exposed cells, and a clear boundary of illumination was present (FIG. 12C). Unlike transmitted light imaging, streptavidin visualization of biotin-PDAB allowed clear 3D reconstruction of the PDAB location with confocal microscopy, demonstrating a cytosolic distribution of the deposited PDAB, albeit with greater PDAB deposition near or in the cell membrane (FIG. 12D). Although miniSOG was not targeted to the cell membrane, it is likely that the PDAB and PANI tended to accumulate within the hydrophobic core of the lipid bilayer due to their high lipophilicity.

Adeno-associated virus (AAV) containing the miniSOG construct was constructed and packaged under the control of the human synapsin promoter for infection of in vitro cultured rat cortical neurons. Neurons were infected with 10¹⁰ vg of AAV (AAVdj) packaged with the miniSOG construct or AAVdj packaged with mCherry as controls (multiplicity of infection [MOI]=1×10⁵) at days in vitro (DIV) 14, followed by 3-5 days of expression post-infection. As in HEK293T cells, irradiation of fixed neurons expressing the intracellular miniSOG in the presence of 1 mM DAB resulted in a dark brown precipitate only in the regions exposed to light by the objective (FIG. 1E). When fixed neurons were instead exposed to light in the presence of the aniline and PPD mixture, the corresponding purple-gray precipitate was observed (FIG. 1F). Neurons expressing only mCherry (hereafter miniSOG−) formed little to no precipitate when irradiated in the presence of either monomer solution (FIGS. 1G-1J).

### Example 2: Biocompatibility of Optogenetic Polymerization

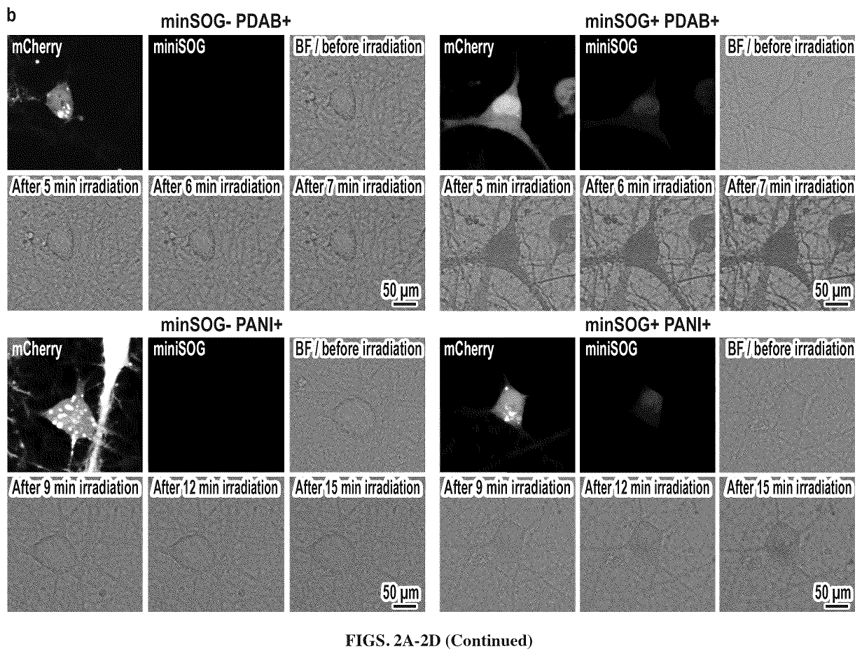

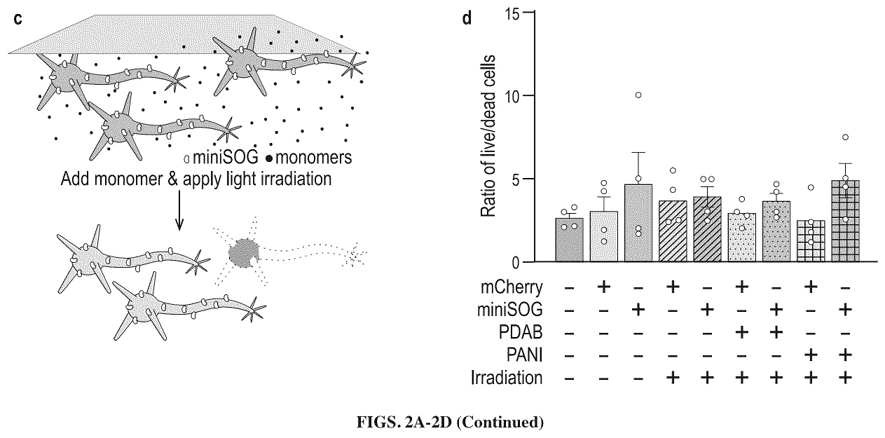

The effects of miniSOG-catalyzed polymerization of conductive and insulating polymers on neuron electrophysiology were examined. First, it was tested if miniSOG− catalyzed polymerization can be achieved effectively and specifically in miniSOG+ neurons for electrophysiological recordings. Specifically, neurons were infected as described in Example 1, but to avoid the potential toxicity of miniSOG (Xu et al., Proc. Natl. Acad. Sci. U.S.A. 109, 7499-7504 (2012)) by achieving a weak expression level, the infection period was restricted to 3-5 days. At DIV 14, the primary neurons showed a pyramidal shape and stellate shape, indicating in vitro maturation (FIGS. 2A-2B). The medium was then replaced with Tyrode's solution containing monomers (1 mM DAB or a mixture of 0.5 mM each of aniline and PPD) and irradiated the neurons with 475 nm blue light for 5-7 minutes to 20 polymerize PDAB or 9-15 minutes to polymerize PANI. Significant precipitates of either DAB or PANI were observed by the obvious darkening of membranes of miniSOG+ neurons but not miniSOG− neurons (control) (FIGS. 2A-2B).

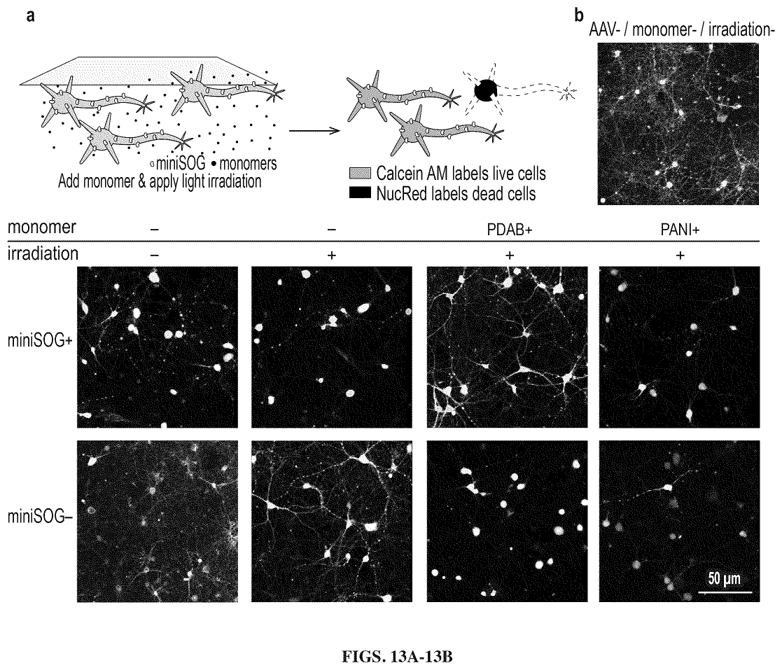

To assess whether the miniSOG-catalyzed polymerization of PANI and PDAB is biocompatible with neurons, cell viability tests were performed and electrophysiology characterization on neurons before and after polymerization. The same polymerization conditions for PDAB and PANI were applied. Specifically, for the cell viability test, Calcein AM and NucRed Probes were added into the medium and incubated for 30 min to stain live and dead neurons, respectively (FIG. 2C). No statistically significant difference was detected between any group, regardless of the presence of miniSOG or each monomer (FIGS. 2D and 13A-13B), indicating that the polymerization reaction as performed here (irradiation for 7 min with approximately 5 mW/mm²) has no acute cytotoxic effects on the viability of neurons.

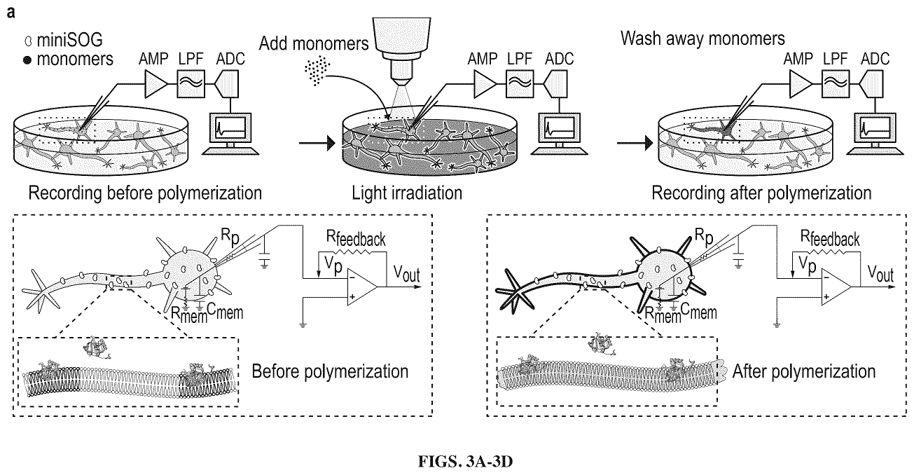

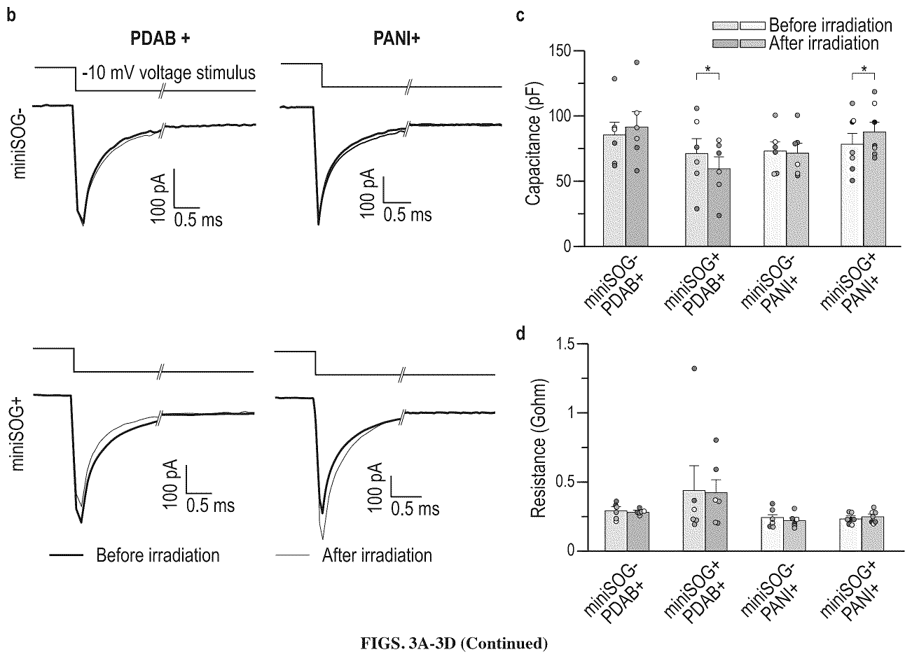

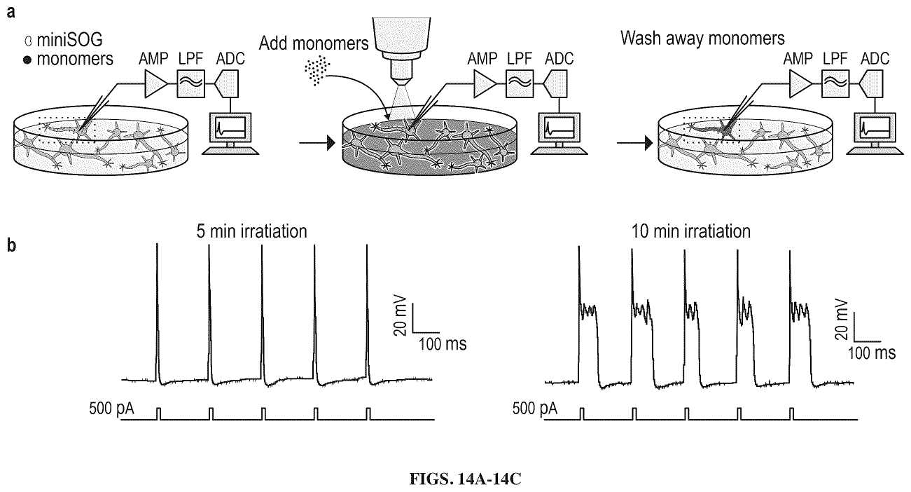

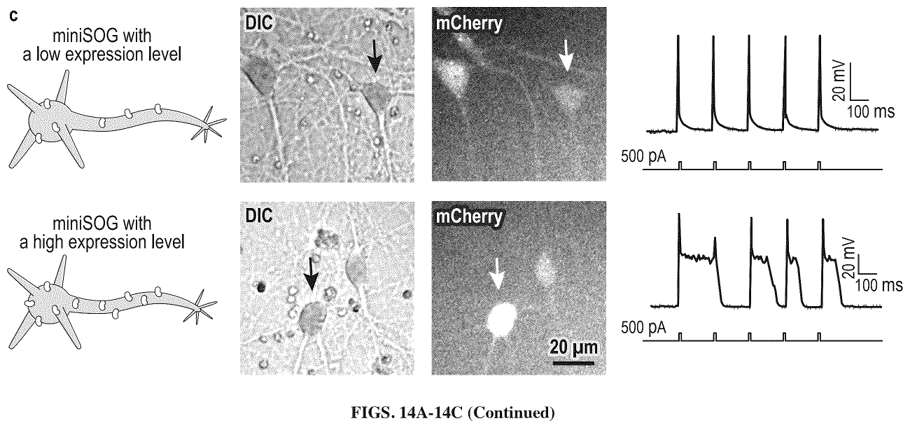

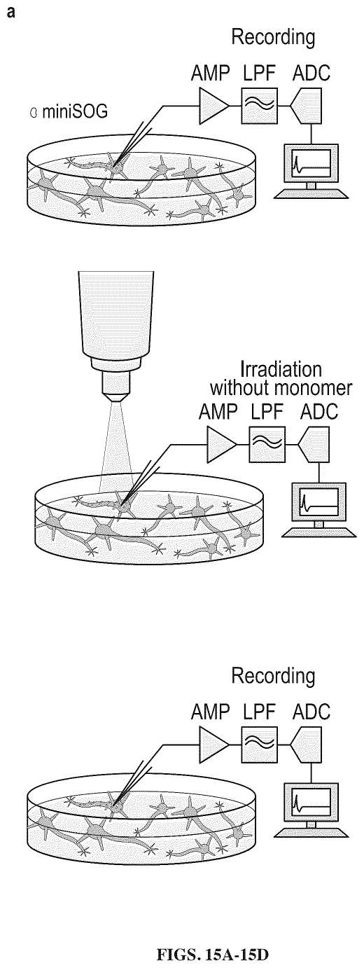

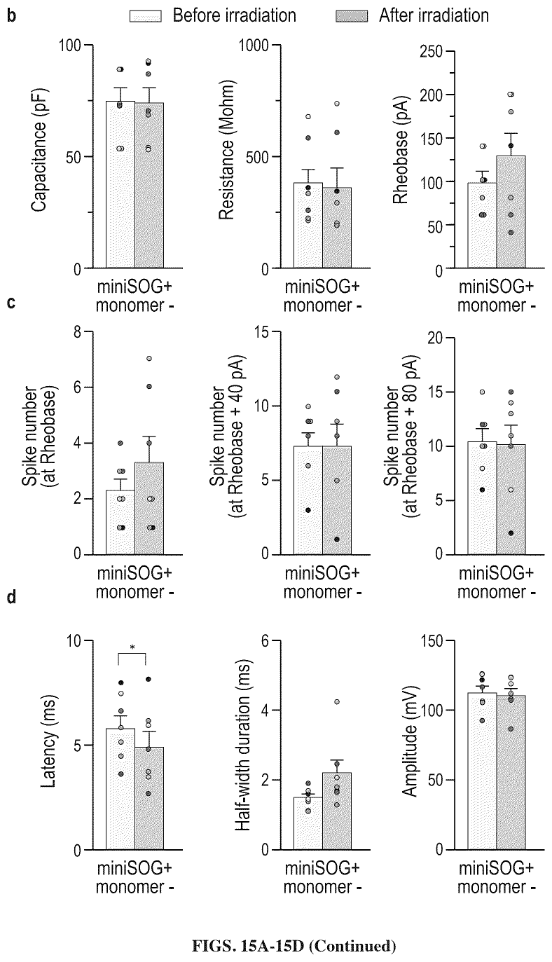

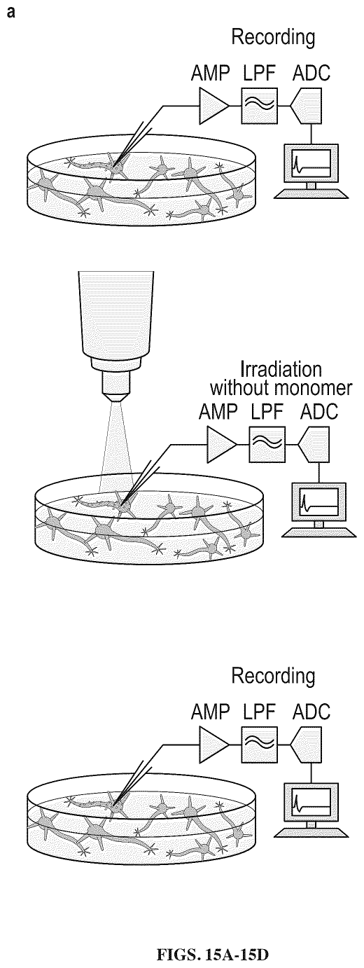

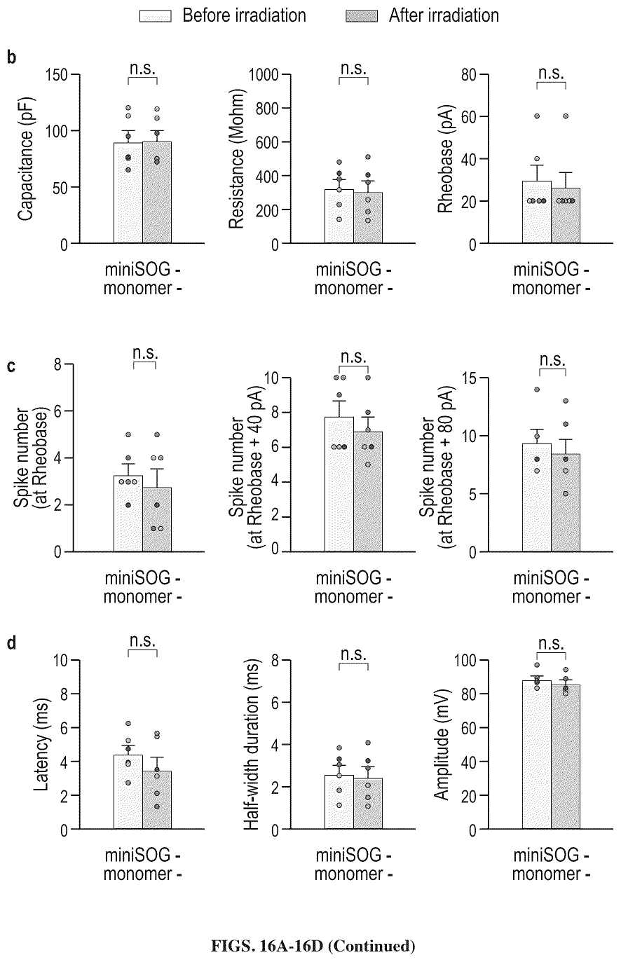

Then, the electrophysiology of the cultured primary rat cortical neurons was characterized before and after the miniSOG-catalyzed polymerization by whole-cell patch-clamp measurement. Notably, compared with a previous Apex2-catalyzed polymerization, the miniSOG-catalyzed polymerization allowed the direct measurement of the same neurons before and after polymerization. During electrophysiological measurements, each neuron was maintained in whole-cell patch mode throughout the entire recording procedure (FIG. 3A). Long-term irradiation of miniSOG itself likely influences neuronal electrophysiological activity. Herein, it was also observed that when the miniSOG expression was high or irradiation duration was long, neurons exhibited hyperexcitability after irradiation in the absence of monomers (FIGS. 14A-14C). Therefore, to avoid the side effects from the irradiation of miniSOG, conditions were tested that would least impact neuronal health and proper functioning before comparing the effects of polymerization on miniSOG+ and miniSOG− neurons. In this measurement, miniSOG+ neurons were recorded in the absence of monomers with differing irradiation time (FIG. 15A). Pyramidal-like neurons with low expression of miniSOG-mCherry were manually selected by fluorescence intensity (corrected total mCherry fluorescence [CTCF]<˜1.3×10⁴ a.u. under −7 mW/mm² green light as in FIG. 14C) and found that up to 7 minutes of 5 mW/mm² blue light irradiation has little-to-no effect on the electrophysiological properties of miniSOG+ neurons (FIGS. 15B-15D). Specifically, comparing the same neurons before and after polymerization, significant changes were not detected in neuronal membrane capacitance (74.59±5.88 pF vs. 74.00±6.50 pF, p>0.05, before vs. after irradiation, mean±s.e.m., unless otherwise stated, n=7), resistance (383.57±63.91 vs. 366.14±85.75 Mohm, n=7, p>0.05), and rheobase (97.14±12.67 vs. 128.57±25.77 pA, n=7, p>0.05) (FIG. 15B). In addition, the action potential spike number at rheobase, rheobase+40 pA, and rheobase+80 pA was not significantly changed (2.5±0.43 vs. 3.7±0.99, 8±0.68 vs. 8.3±1.2, and 11.2±0.98 vs. 11.5±1.3 pA,n=7, p>0.05), suggesting no alterations in cellular excitability (FIG. 15C). Furthermore, the half-width duration and amplitude of current-elicited action potentials were not significantly altered before and after irradiation (half-width duration: 1.49±0.11 ms vs 2.19±0.37 ms; amplitude: 112.25±4.71 mV vs 110.32±4.84 mV, n=7, p>0.05) (FIGS. 15C-15D). However, the latency of phasic stimulation-induced action potentials was significantly decreased (5.87±0.60 ms vs 4.99±0.72 ms, n=7, p=0.038), suggesting that there could be some unanticipated effects resulting from miniSOG irradiation. Despite some effects on latency, these results demonstrate that 7 min of 5 mW/mm², 475 nm light irradiation of neurons with controlled miniSOG expression will not generally impact neuron electrophysiological behaviors as measured by the whole-cell patch-clamp. Although the PDAB precipitate formed under these weak expression and irradiation conditions is challenging to visualize by transmitted light imaging, streptavidin staining of miniSOG+ neurons polymerized with biotin-DAB under these conditions revealed robust PDAB deposition (FIGS. 12E-12I). Similarly, when these irradiation conditions were applied to miniSOG− neurons, no significant changes in any of the above electrophysiological properties were observed (FIG. 16A-16D). Therefore, this AAVdj infection and optical irradiation condition was applied to the optogenetic polymerization experiments herein (FIG. 3A).

### Example 3: Modulation of Neuron Excitability by Optogenetic Polymerization

Next, it was examined whether the miniSOG-catalyzed polymerization of PDAB and PANI could alter the electrophysiological behaviors of neurons. Specifically, the inclusion of conductive or insulating polymers on and within the cell membrane is likely to increase or decrease the capacitance of the membrane, respectively, which can be modeled as

$$C = ∈ \frac{A}{d}$$

in which C, ∈, A, and d are the membrane capacitance, permittivity, surface area, and distance of separation, respectively. In the case of conductive polymers (e.g., PANI), the large permittivity of these materials (about 10⁴) would be expected to increase the permittivity of the membrane upon assembly within the membrane, increasing the capacitance accordingly. In contrast, the incorporation of insulating polymers (e.g., PDAB), which have a much lower permittivity, would lead to a decrease in capacitance upon incorporation within the membrane. Note that PDAB accumulates outside of membranes, which would primarily modulate d, but this would also lead to decreases in capacitance in accordance with increasing membrane thickness. According to the Hodgkin-Huxley model, membrane capacitance can be regarded as an inversely proportional scale factor that influences the sensitivity of the changes in membrane potential in response to external stimuli. These increases or decreases in membrane capacitance are then reflected as decreases or increases in neuron excitability, respectively.

These optogenetic polymerization and assembly-induced changes in membrane properties were characterized using whole-cell patch-clamp recordings. The above light irradiation conditions were applied to neurons recorded under whole-cell patch mode in the presence of either the PDAB or PANI precursor solutions, permitting electrophysiological characterization of the same neuron before and after polymer assembly. To avoid any side effects on the membrane properties from remaining monomers after optogenetic polymerization, cell electrophysiology was measured after removing the monomer solution. Directly following the light irradiation, pronounced changes were observed in the square voltage step-induced current waveform from miniSOG+ neurons, but no change from miniSOG− neurons (FIG. 3B). Strikingly, irradiated miniSOG+/PDAB neurons had a significant decrease in membrane capacitance (71.33±11.41 pF vs 59.93±8.91 pF, n=6,p=0.027) whereas irradiated miniSOG+/PANI neurons had a significant increase in membrane capacitance (78.82±8.33 pF vs 88.11±7.56 pF, n=7, p=0.021). Importantly, no statistically significant changes of membrane capacitance were detected from irradiated miniSOG−/PDAB (85.42±9.97 pF vs 91.57±11.53 pF, n=6, p>0.05) and miniSOG−/PANI neurons (73.62±6.90 pF vs 72.15±7.29 pF, n=6, p>0.05) (FIGS. 3B-3C). Meanwhile, a significant change of the membrane resistance was not observed from all neurons after optical irradiation, indicating that light exposure or the resulting polymerization in these conditions does not alter whole-cell conductance (FIGS. 3B and 3D).

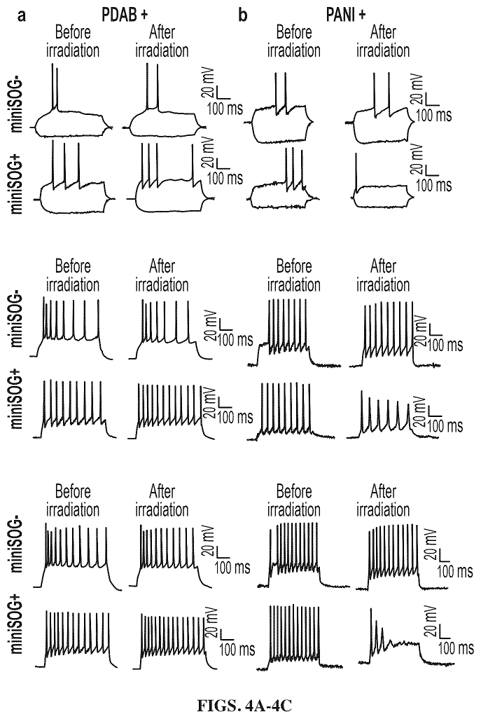

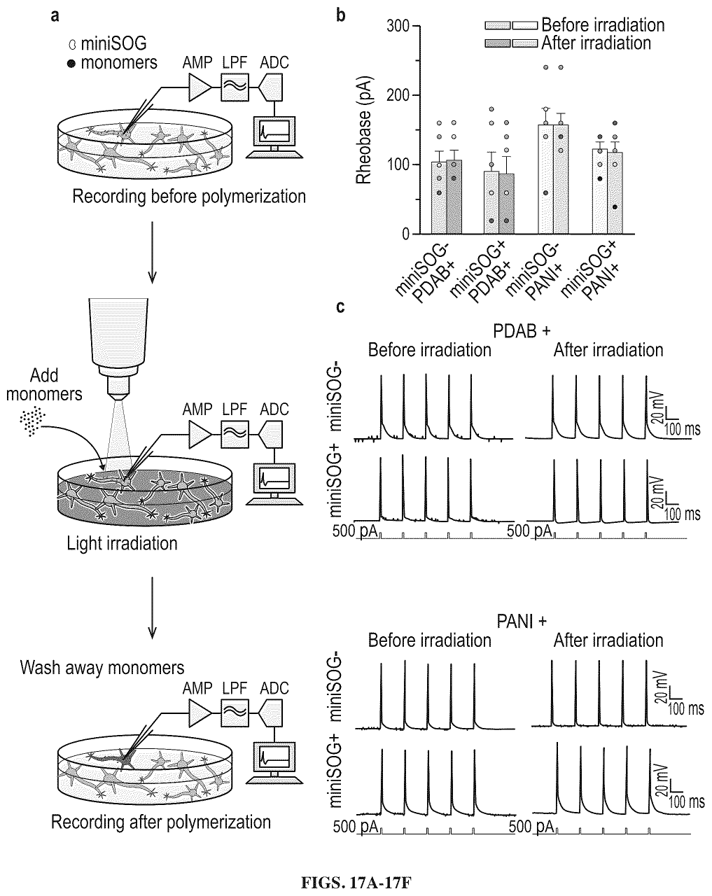

Whether the optogenetic polymerization can increase or decrease the intrinsic neuronal excitability was examined, in accordance with the changes in membrane capacitance. The irradiated miniSOG+/PDAB neurons exhibited increased current injection-evoked action potential firing to depolarizing stimuli (the spike number increased from 2.33±0.61 to 4.83±1.19 at rheobase, p=0.037, from 6.33±0.80 to 8.50±0.92 at rheobase+40 pA, p=0.010, and from 8.17±1.40 to 11.17±0.75 at rheobase+80 pA, p=0.023, n=6) (FIGS. 4A and 4C), whereas the irradiated miniSOG+/PANI neurons exhibited decreased action potential firing to depolarizing stimuli (the spike number decreased from 1.71±0.36 to 1.57±0.92 at rheobase, p=0.90, from 5.57±0.95 to 1.57±0.37 at rheobase+40 pA, p=0.0064, and from 8.00±0.72 to 1.86±0.55 at rheobase+80 pA, p=0.0013, n=7) (FIGS. 4B-4C). In contrast, the irradiated miniSOG−/PDAB and miniSOG−/PANI did not result in a significant change in the current injection-evoked spike numbers. Notably, no changes were observed in the average rheobase level (current spiking threshold) from all samples (FIG. 17B), consistent with the lack of an effect on membrane resistance. Collectively, these results demonstrate that optogenetic PDAB and PANI assembly can elicit both increases and decreases in the intrinsic excitability of neurons.

The stimulation-elicited intracellular action potentials of neurons were systematically characterized before and after optogenetic polymerization by applying brief phasic current stimulation. No significant changes were detected in the amplitude and kinetics of phasic current-evoked responses after PDAB polymerization (FIGS. 17C-17F). However, a significant increase was observed of the spike half-width duration (from 1.61±0.09 ms to 2.55±0.24 ms, n=7, p=0.0087) in miniSOG+/PANI group while the miniSOG−/PANI group did not show a statistically significant change (1.47±0.14 ms vs. 1.49±0.19, n=6, p>0.05) (FIG. 17F). The increase of action potential spike half-width suggests the decrease of neuronal excitability as a result of decreased depolarization and repolarization slopes.

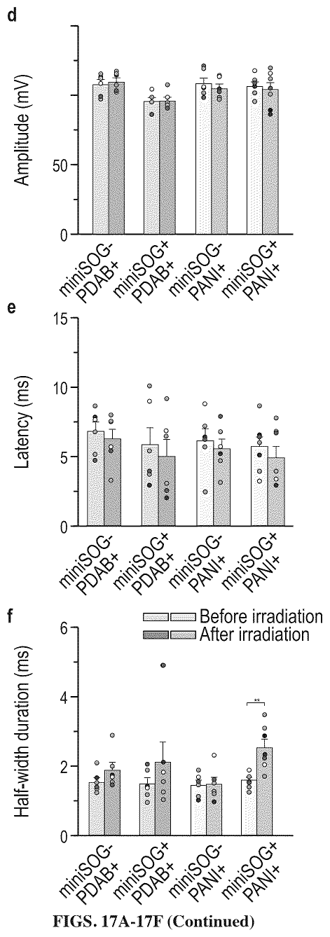

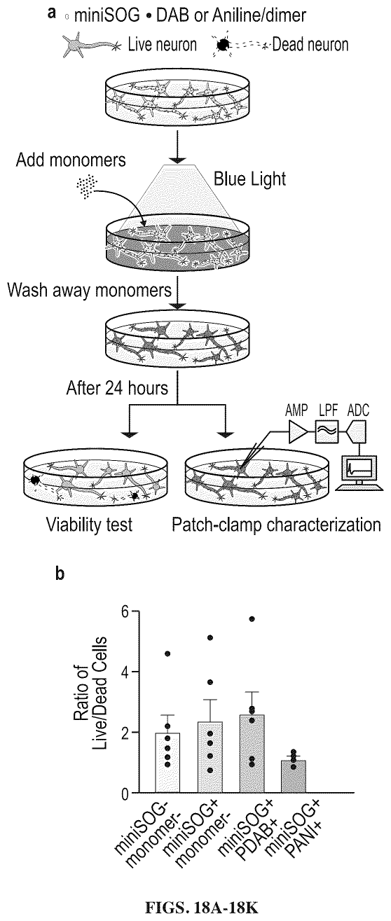

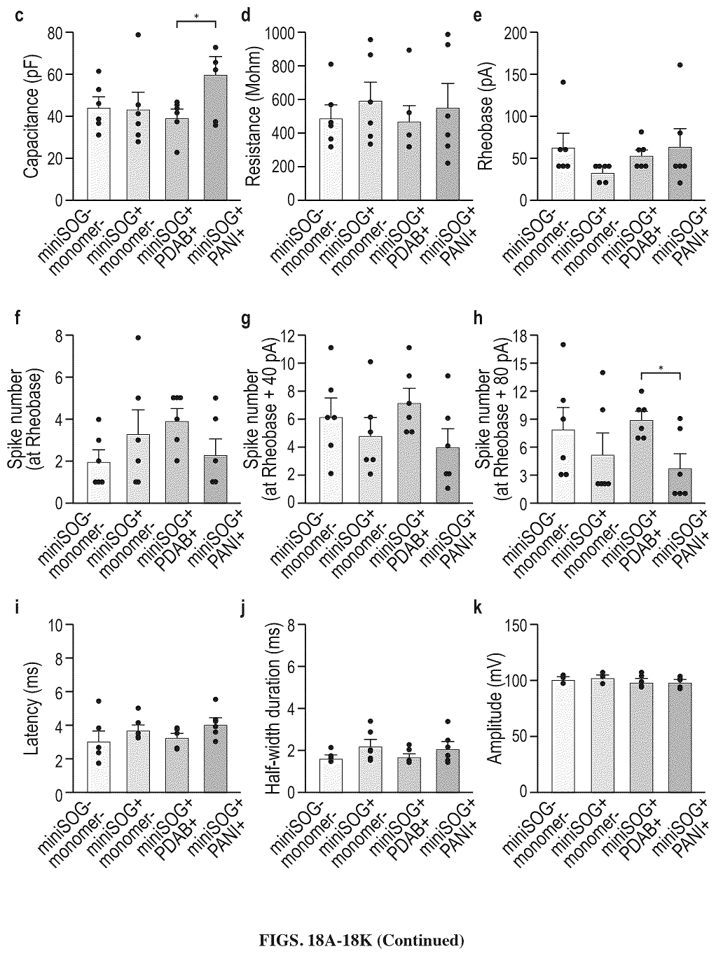

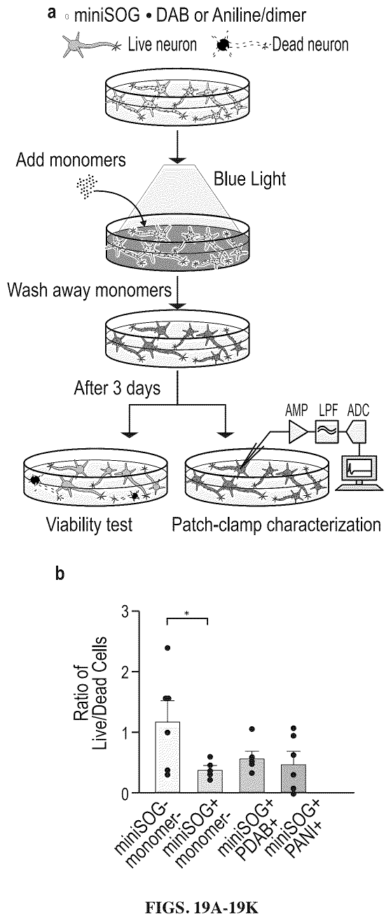

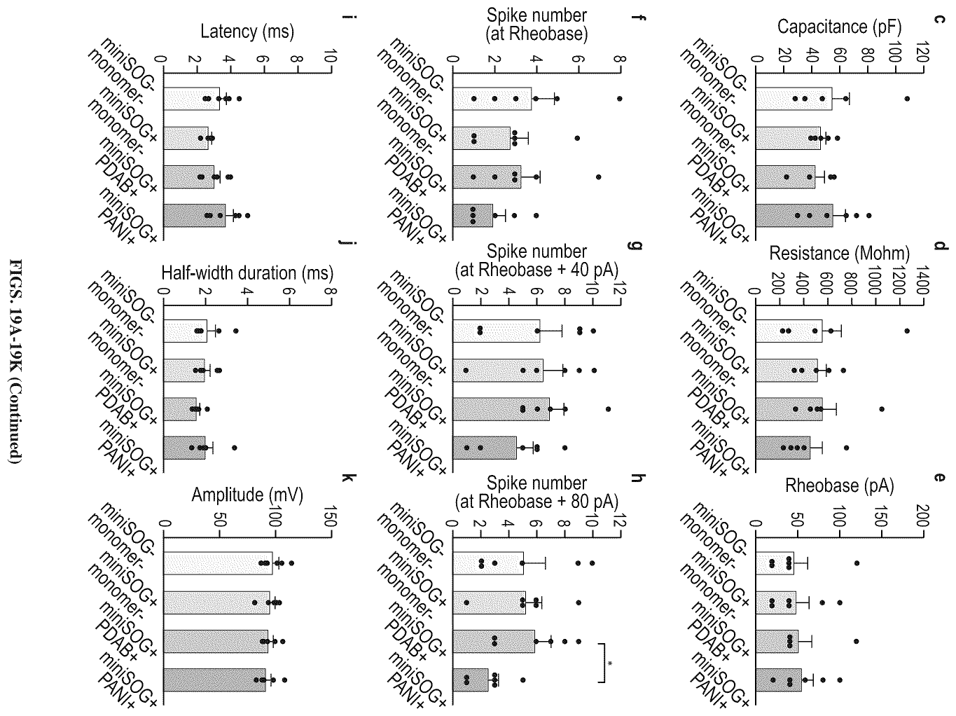

Next, the long-term effects of PDAB and PANI polymerization were characterized on neuron viability and electrophysiology, at both 1 and 3 days post-polymerization. Because the neurons cannot be held under whole-cell patch for this duration, the electrophysiology was instead compared between miniSOG−/monomer−, miniSOG+/monomer−, miniSOG+/PDAB, and miniSOG+/PANI neurons at the population level at each time point post-polymerization (FIGS. 18A and 19A). Viability of miniSOG+/PDAB and miniSOG+/PANI neurons was not significantly impacted at either 1 or 3 days post-polymerization, although the viability of miniSOG+/monomer− neurons did decrease significantly after 3 days, likely due to the absence of singlet oxygen quenching by the monomers (FIGS. 18B and 19B). Patch-clamp recordings revealed significantly higher capacitance in the miniSOG+/PANI neurons compared to miniSOG+/PDAB neurons at 1 day post-polymerization (60.11±8.20 pF vs 39.75±3.68 pF, n=6, p=0.047, unpaired two tailed t-test) without significant changes in resistance or rheobase (FIGS. 18C-18E). At 3 days post-polymerization, the overall trend in capacitance was retained, albeit not statistically significant (55.79±8.14 pF vs 43.66±5.53 pF, n=6, p=0.246, unpaired two tailed t-test) with no clear differences in resistance or rheobase (FIGS. 19C-19E).

In terms of excitability, the spike number when held at rheobase+80 pA was significantly lower in the miniSOG+/PANI neurons compared to miniSOG+/PDAB neurons at both 1 day post-polymerization (3.83±1.52 vs 9.00±0.82, n=6, p=0.013, unpaired two-tailed t-test) and 3 days post-polymerization (2.667±0.61 vs 6.00±1.03, n=6, p=0.020, unpaired two-tailed t-test), while at rheobase and rheobase+40 pA, the trend was the same, but not statistically significant at either time point (FIGS. 18F-18H and 19F-19H). No statistically significant differences in spike latency, half-width duration, or amplitude were observed between any groups at either time point (FIGS. 18I-18K and 19I-19K). These results indicate that optogenetic PDAB and PANI polymerization are capable of capacitance-induced changes in neuron excitability lasting days after polymerization, without impacting neuron viability. Additionally, not wishing to be bound by theory, it is believed that the long-term stability of these electrophysiological changes suggests that the polymers tend to accumulate within the membrane itself, as opposed to on the surface of the membrane, where gradual diffusion throughout the cytosol would be expected to reverse the effects of optogenetic polymerization over time.

### Example 4: Stepwise Control of Optogenetic Polymerization and Neuron Excitability

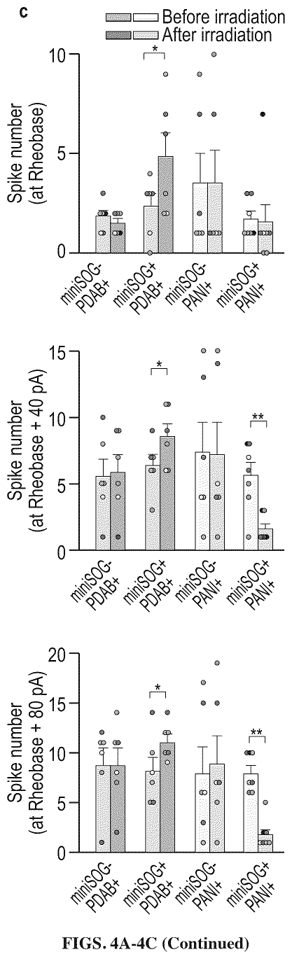

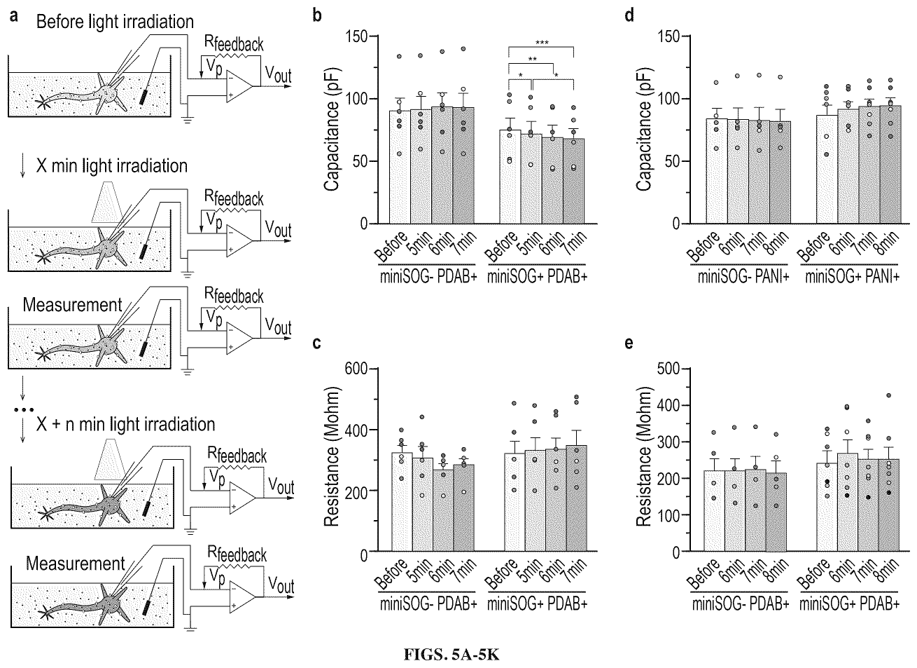

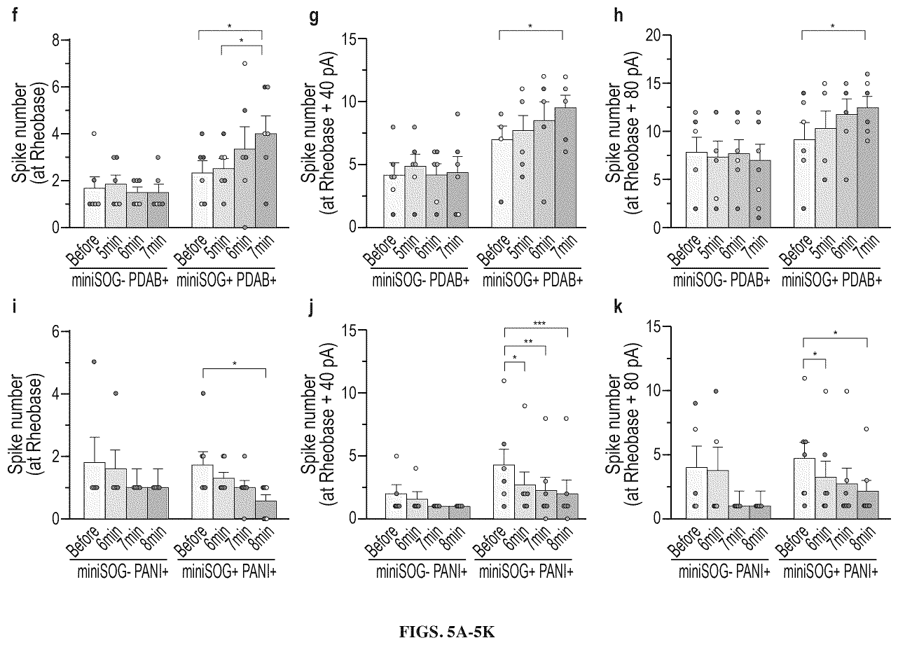

Optogenetic polymerization can provide fine temporal control over the polymerization reaction, thus potentially allowing for stepwise fine-tuning of membrane properties by controlling the length of light exposure. To demonstrate that the optogenetic polymerization can be controlled in a predictable, stepwise manner, the stepwise change of the membrane properties was measured by continuously measuring the cultured primary neurons in the monomer solution during iterative optogenetic polymerization. After characterizing the initial membrane properties and electrophysiological behaviors of the neurons, the membrane properties and electrophysiological behaviors were sequentially measured of the same neurons after 5, 6, and 7 minutes of irradiation (FIG. 5A). It is noteworthy that, unlike the aforementioned single-timepoint measurements, the stepwise changes were recorded in the presence of monomers due to the difficulty of washing and re-perfusing the monomer with each measurement. The capacitance of miniSOG+ neurons were decreased in a stepwise manner during the PDAB polymerization (miniSOG+/PDAB, before irradiation: 74.98±9.08 pF, 5-min irradiation: 72.12±9.10 pF, p=0.013 vs before irradiation; 6-min irradiation: 69.31±9.03 pF, p=0.005 vs before irradiation; 7-min irradiation: 67.35±8.15 pF, p=0.009 vs before irradiation; n=6) and increased in a stepwise manner during the PANI polymerization (miniSOGI+/PANI, before irradiation: 87.21±7.97 pF, 6-min irradiation: 91.80±5.73 pF, p=0.22 vs before; 7-min irradiation: 94.04±5.93 pF, p=0.067 vs before; 8-min irradiation: 95.03±5.97 pF, p=0.058 vs before; n=7) (FIGS. 5B and 5D). However, this trend is not statistically significant in the case of PANI, likely due to the transient effects of the monomer solution itself on neuron electrophysiology. Similar to the results herein, the membrane resistances of irradiated miniSOG−/PDAB and miniSOG−/PANI neurons did not show significant changes over time (FIGS. 5C and 5E).

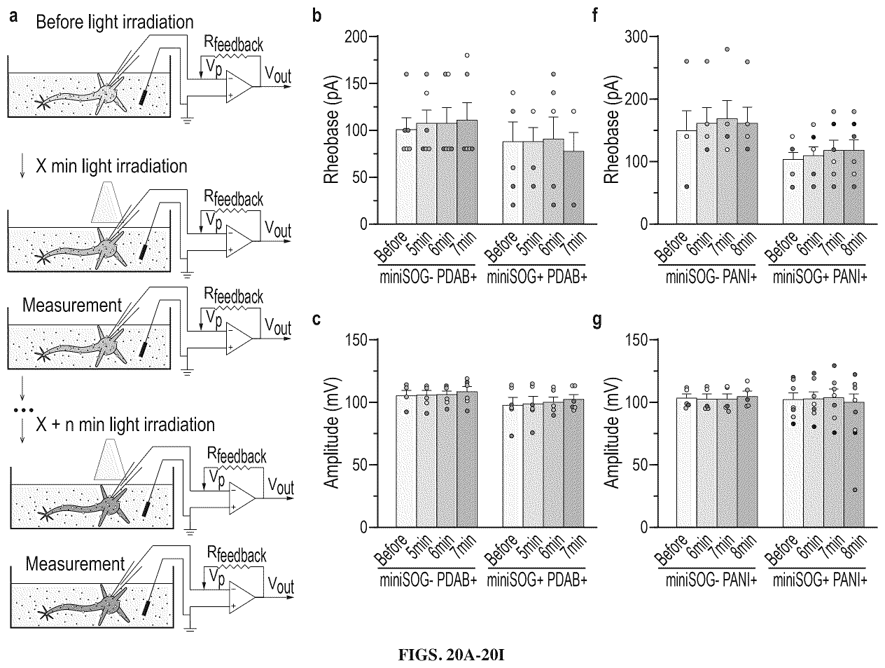

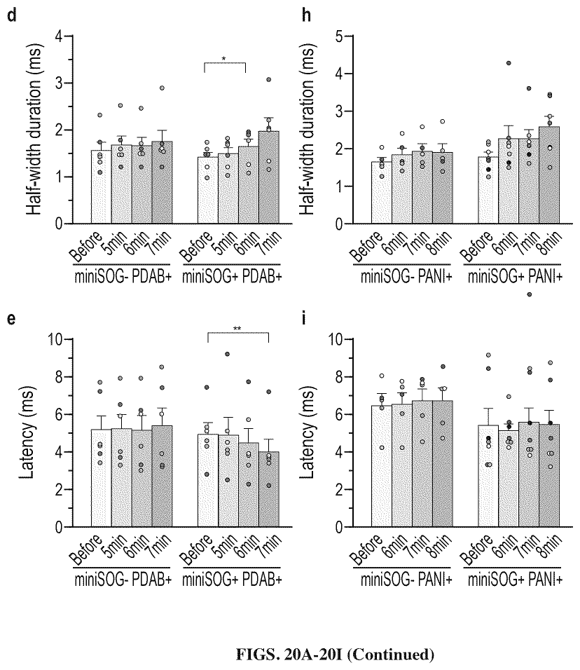

The spike numbers of irradiated miniSOG+/PDAB neurons also increased significantly during the stepwise PDAB polymerization at the rheobase level (miniSOG+/DAB, before irradiation: 2.33±0.49, 5-min irradiation: 2.5±0.43, p=0.77 vs before; 6-min irradiation: 3.33±0.99, p=0.45 vs before; 7-min irradiation: 4.00±0.77, p=0.03 vs before; n=6), at rheobase level+40 pA (miniSOG+/DAB, before irradiation: 7.00±1.07, 5-min irradiation: 7.67±1.23, p=0.52 vs before; 6-min irradiation: 8.50±1.46, p=0.39 vs before; 7-min irradiation: 9.50±0.99, p=0.042 vs before; n=6) and at rheobase level+80 pA (miniSOG+/DAB, before irradiation: 9.17±1.82, 5-min irradiation: 10.33±1.82, p=0.20 vs before; 6-min irradiation: 11.83±1.58, p=0.20 vs before; 7-min irradiation: 12.50±1.18, p=0.045 vs before; n=6) (FIGS. 5F-5H). Conversely, the spike numbers of irradiated miniSOG+/PANI neurons showed a stepwise decrease during the PANI polymerization at rheobase level (miniSOG+/PANI, before irradiation: 1.71±0.42; 6-min irradiation: 1.29±0.18, p=0.36 vs before; 7-min irradiation: 1.00±0.22, p=0.14 vs before; 8-min irradiation: 0.57±0.20, p=0.030 vs before; n=7), at rheobase level+40 pA (miniSOG+/PANI, before irradiation: 4.29±1.27; 6-min irradiation: 2.71±1.06, p=0.025 vs before; 7-min irradiation: 2.29±1.02, p=0.033 vs before; 8-min irradiation: 2.00±1.02, p=0.0068 vs before; n=7) and at rheobase level+80 pA (miniSOG+/PANI, before: 4.71±1.30, 6-min irradiation: 3.29±1.23, p=0.046 vs before; 7-min irradiation: 2.71±1.25, p=0.068 vs before; 8-min irradiation: 2.14±0.86, p=0.035 vs before; n=7) (FIGS. 5I-5K). Consistent with the results herein, the irradiated miniSOG−/PDAB or miniSOG−/PANI control neurons did not show significant changes for all measures (FIGS. 5F-5K) and the membrane resistance or rheobase did not change significantly in miniSOG+ and miniSOG− neurons during PDAB or PANI polymerization (FIGS. 5C, 5E, 20B, and 20F). Additionally, the phasic current-induced response after stepwise PDAB polymerization showed no changes on the evoked action potential spike amplitude (FIG. 20C). However, the latency decreased from 4.91±0.61 ms (before irradiation) to 4.88±0.93 ms (p=0.93 vs before), 4.45±0.79 ms (p=0.15 vs before), and 3.98±0.69 ms (p=0.007 vs before) after 5-, 6-, and 7-min irradiation (n=6) (FIG. 20E), which suggests a higher likelihood to elicit action potentials due to increased neuronal excitability after PDAB polymerization. Meanwhile, the half-width duration increased from 1.42±0.11 ms (before irradiation) to 1.50±0.13 ms (p=0.086 vs before), 1.66±0.15 ms (p=0.028 vs before), and 1.98±0.28 ms (p=0.052 vs before) after 5-, 6-, and 7-min irradiation (n=6) (FIG. 20E). No significant changes were detected in the amplitude or kinetics of phasic current-evoked action potential after stepwise PANI polymerization on both miniSOG+ and miniSOG− neurons (FIGS. 20G-20I). Together, these results demonstrate the ability to modulate membrane capacitance and excitability stepwise by iterative optogenetic polymerization of conductive and insulating polymers.

This disclosure provides a new method for modulating neural activity was introduced using optogenetic polymerization and assembly of electroactive polymers on specified cellular membranes. Compared with the previous neuromodulation method based on peroxidase catalysis, this optogenetic polymerization strategy provided spatiotemporal control over the polymer generation and assembly and reduces the broad toxicity of hydrogen peroxide. Furthermore, extending this photosensitized chemical assembly strategy to genetically targetable photosensitizers, such as Fluorogen Activating Protein Targeted and Activated Photosensitizer (FAP-TAPs), which are described by He et al. (Nat Methods. 2016 March; 13(3): 263-268), which is incorporated herein in its entirety, with varying excitation wavelengths potentially allows for simultaneous, multicolor manipulation of different types of cells in intact neural circuits. Continued development of genetically targetable photosensitizers could enable polymer assembly with subcellular specificity and lower diffuse cytotoxicity. Engineering the structures of the monomers and oligomers can also further improve the biocompatibility and extend their functionalities, including potential degradability. These developments in biocompatible, in vivo polymer assembly are expected to allow this technique to be applied to in vivo manipulation of neuronal circuits and animal behaviors.

Notably, in contrast to the transient modulation of neuron activities achieved by manipulating ion channel conductivities, materials-based manipulation of membrane capacitance can modulate the neuronal electrophysiology in a long-term stable manner, at a time scale relevant to brain development, learning, and aging. Future development of photosensitized in situ biosynthesis of functional polymers in vivo could enable bidirectional neuromodulation to control brain activities and freely behaving animal behaviors such as precisely tuning the intrinsic excitation-inhibition balance within cortical microcircuits over longer timescales. Furthermore, it may ultimately provide electrotherapeutic stimulation options to ameliorate neurodegenerative and myelination degenerative diseases in a long-term stable manner through the restoration of cell excitability and action potential propagation. Finally, the advances in biocompatible functional polymer synthesis presented herein demonstrate the utility of in situ nanomaterial synthesis and assembly as an emerging synthetic biology technique for interfacing biological systems with synthetic materials. This can potentially provide far greater control over the integration of these materials at the cellular level than is typically afforded by conventional, prefabricated nanomaterials, potentially enabling a new generation of synthetic biology techniques.

The results described above were obtained using the following materials and methods.

### Photopolymerization in Living and Fixed HEK293T Cells

HEK293T cells were seeded onto glass coverslips pre-coated with Matrigel® according to the manufacturer's protocol (available at: www.coming.com/worldwide/en/products/life-sciences/resources/webforms/the-ultimate-guide-to-corning-matrigel-matrix.html) and grown in DMEM with 10% FBS in a 5% CO₂ environment to approximately 90% confluence before transfection. Cells were transfected with either the miniSOG plasmid or the SuperNova Green plasmid under the control of the CAG promoter using Lipofectamine™ 3000 based on the manufacturer's protocol (available at: www.thermofisher.com/document-connect/document-connect.html?url=https %3A %2F %2Fassets.thermofisher.com %2FTFS-Assets %2FLSG %2Fmanuals %2Flipofectamine3000_protocol.pdf), then cultured for an additional 24 hours. For living cell experiments, the cells were stained with 10 μM Hoechst 33342 (Thermo Fisher) for 10 minutes in FluoroBrite™ DMEM, rinsed with PBS, then kept in FluoroBrite™ DMEM until polymerization. During the polymerization, the FluoroBrite™ DMEM was removed from the well and replaced with the working solution of monomer approximately 5 min prior to irradiation. Confocal fluorescence and transmitted light (TL) images were captured before and after exposure to 475 nm light for 5-10 min. For routine photopolymerization, the epifluorescence light source on the GFP filter set was used with the intensity adjusted to approximately 62 mW/mm².

For fixed cell experiments, the cells were stained with Hoechst as above, rinsed with PBS, then fixed in 100 mM sodium cacodylate, with 3.2% paraformaldehyde and 0.25% glutaraldehyde (Electron Microscopy Sciences) at pH 7.4 for 15 min before rinsing twice with PBS. Cells were then quenched in 50 mM glycine in PBS for 15 min before rinsing twice again in PBS. The working solution of monomer was prepared as above, except in PBS, then was added to the cells before performing photopolymerization. Working solutions of the monomer were prepared by dissolving 1 mM DAB or 0.75-1 mM aniline+0.75-1 mM PPD in FluoroBrite™ DMEM, pre-warmed and equilibrated in the 5% CO₂ environment, then filtering through a 0.22 μM syringe filter before adding to cells. Stock solutions of DAB or aniline were prepared by dissolving DAB tetrahydrochloride hydrate (Sigma Aldrich) or aniline (Sigma Aldrich) to 100 mM in ultrapure water, while the stock solution of PPD (Sigma Aldrich) was prepared by suspending it to 100 mM in 150 mM HCl. The stock solution was stirred and periodically sonicated at room temperature for 1-2 hours to create a fine green-gray suspension that dissolved completely upon dilution into the working solution.

### Photopolymerization in Living and Fixed Neurons

### Primary Neurons Culture and Transfection

Primary cultures of cortical rat neurons were prepared as follows, following IACUC guidelines. The cortex of Sprague-Dawley rat pups was removed at embryonic day 17 (E17). Cortexes were digested with 0.4 mg/mL papain and plated onto 12 mm glass coverslips pre-coated with 1:80 Matrigel (Corning). Cells were plated in 24-well plates at a density of 100,000 cells per well. The cultured neurons were maintained in NbActiv4 medium (BrainBits) and kept in a humid culture incubator with 5% CO₂ at 37° C. Primary culture neurons were infected with 1×10¹⁰ vg AAVdj-hSyn-miniSOG-T2A-mCherry or AAVdj-hSyn-mCherry at 14 days in vitro (DIV). After 3-5 days of expression, photopolymerization was performed according to the above procedures for HEK293T cells.

### Biotin-DAB Photopolymerization and Streptavidin-AF647 Staining

MiniSOG+ HEK293T cells or neurons were fixed and glycine-quenched as described above, then permeabilized in 0.1% Triton X-100 in PBS for 15 min, blocked with a streptavidin/biotin blocking kit according to manufacturer's instructions (Thermo Fisher, cat.E21390), then polymerized as described above for DAB, in the presence of 1 mM biotin-DAB instead for 10 min at ˜155 mW/mm² of 475 nm light. The same procedure was used for neurons, except the intensity was reduced to ˜3.2 mW/mm². Following polymerization, cells were washed 3×5 min with PBS+0.1% Tween-20, then stained with a 1:750 dilution of streptavidin-AF647 (Thermo Fisher, cat.S21374) in PBS/Tween for 90 min, followed by 3×20 min washes of PBS/Tween prior to confocal imaging.

### UV/Vis Spectroscopy of In Situ Polymerized PANI

HEK293T cells were cultured and transfected on Aclar® coverslips as above. Hoechst staining was skipped, and cells were fixed, blocked, and the monomer solution was added as above. In order to maximize the area exposed to light, polymerization was performed on the 4× objective of an epifluorescence microscope, using a 475 nm laser with an approximate intensity of 50 mW/mm². Cells were then rinsed several times with ultrapure water and air-dried for several hours before acquiring UV/vis spectra on a Cary 60 UV/vis spectrophotometer. For acid doping, the cover slip was kept in a sealed chamber with a few drops of concentrated hydrochloric acid added to the bottom for 1 hour.

Photooxidation of PPD with Small Molecule Photosensitizers

A 100 mM suspension of PPD in 150 mM HCl was prepared as described above, then diluted to 500 μM in PBS. Methylene blue (Sigma Aldrich) or Victoria blue BO (Sigma Aldrich) was added to 5 μM final concentration along with 5 mM sodium azide (Sigma Aldrich) or 45 μg/mL superoxide dismutase (from bovine erythrocytes; Sigma Aldrich) from freshly prepared aqueous stock solutions. Reactions were monitored in an untreated, glass-bottom 96-well plate. UV/vis spectra were acquired on a PerkinElmer EnSpire® plate reader prior to irradiation of the entire well using a 10× microscope objective on the Cy5 filter set (˜108 mW). After 30 seconds, no precipitate was visible, and the UV/vis spectra was measured again, before continuing irradiation for an additional minute until a dark precipitate began to form in the wells containing methylene blue.

### Molecular Cloning, Viral Vector Construction, and Virus Production

For HEK293T expression, the miniSOG coding sequence, which is described, for example, by Shu et al., PLoS Biol. 2011 April; 9(4):e1001041, which is incorporated herein by reference in its entirety, was inserted into a pAAV-CAG-T2A-dTomato vector backbone, which is commercially available from Addgene, and is described, for example, by Kuljis et al., eNeuro 2019 Oct. 31; 6(5):ENEURO.0193-19.2019, for cytosolic expression under the CAG promoter using NEBuilder® HiFi DNA assembly. For a superoxide-producing control, the codon optimized coding sequence of the Killer Red derivative SuperNova Green, synthesized by Genscript, was inserted instead. For AAV production, the same miniSOG sequence was cloned into a pAAV-hSyn-T2A-mCherry backbone.

The following AAV viral vectors under the control of the human Synapsin (hSyn) promoter were packaged as AAVdj.
(1) pAAVdj-hSyn-mCherry
(2) pAAVdj-hSyn-miniSOG-T2A-mCherry

### Electrophysiological Characterization

### Whole-Cell Patch Clamp

For whole-cell recording, electrophysiological parameters of cultured neurons were amplified and digitized using the Multiclamp 700B and DigiDatal400 (Molecular Devices) and pipettes with a resistance of 4-6 MOhm filled with an internal solution containing: 128 mM K-gluconate, 10 mM Na-phosphocreatine, 10 mM HEPES, 1.1 mM EGTA, 5 mM ATP-Mg, 0.4 mM GTP-Na with the pH adjusted to 7.4 with KOH. The osmolarity of the internal solution was adjusted to around 300 mOsm with sucrose. The neurons infected with AAVdj-hsyn-mCherry or AAVdj-hsyn-miniSOG-T2A-mCherry cultured on glass coverslips were exposed to Tyrode's solution (150 mM NaCl, 4 mM KCl, 2 mM MgCl₂, 2 mM CaCl₂), 20 mM glucose, 10 mM HEPES; titrated to pH 7.35 with NaOH and adjusted osmolarity to 320-330). 2, 3-Dioxo-6-nitro-1,2,3,4-tetrahydrobenzo[f]quinoxaline-7-sulfonamide disodium salt (NBQX; 20 μM, Tocris) as the AMPA receptor blocker and D-2-amino-5-phosphono-valeric acid (D-AP5; 25 uM, Tocris) as the NMDA receptor blocker were contained in all bath solutions. The cells were held in voltage-clamp mode at −75 mV. Membrane resistance and cellular capacitance were calculated from a 10-mV depolarization step in voltage clamp. Then, the mode was switched to current clamp followed by stepwise tonic current injection (20 pA per step from −100 pA to 280 pA) to elicit action potentials and to determine rheobases. Phasic currents (500 pA, 10 ms, 5 Hz) were injected to generate action potentials while holding at −70 to −75 mV membrane potential. Then the bath solution was switched to the Tyrode's solution containing 1 mM DAB or 0.5 mM aniline+0.5 mM PPD with NBQX and D-AP5 at pH 7.35 and osmolarity 320-330. Recording was repeated after neurons were completely immersed into Tyrode's solution with monomers and after progressively extended blue light irradiation (5 minutes, 6 minutes and 7 minutes stepwise, ˜5 mW/mm²) to induce polymerization. Following the end of polymerization, monomers were washed away by replacing the bath solution with Tyrode's solution without monomers and recording procedure was repeated. FIGS. 3A-3D, 4A-4D, and 15A-15D compared the electrophysiological properties between recorded parameters before adding monomers and washing away monomers. FIGS. 5A-5K and 17A-17F compared the change of electrophysiological properties when neurons were immersed in the Tyrode's solution containing monomers. FIGS. 14A-14C compared the electrophysiological properties in the absence of monomers by immersing the neurons in the monomer-free Tyrode's solution during recordings. Analyses of physiological results were performed using ClampFit software (Axon Instruments). Spike width was estimated at the half-peak position from the threshold potential to the peak of a single action potential. Spike latency was estimated as the time point of the peak of an action potential starting from the onset of the current injection. Spike amplitude was estimated as the magnitude of the action potential, taking the resting potential as the baseline. Spike number was estimated by averaging the spike numbers counted at the rheobase, rheobase+40 pA and rheobase+80 pA.

### Cell Viability Assay

2×10⁴ primary neurons were added in the Nb4 medium and seeded into each well of a 96-well-plate. All the wells were divided into 9 groups. They were set as follows: 1. AAV−, monomer−, and irradiation− (Baseline control); 2. mCherry+, monomer−, and irradiation− (Test effect of AAV infection); 3. miniSOG+, monomer−, irradiation− (Test effect of AAV and miniSOG infection); 4. mCherry+, DAB−, and 7 min irradiation; 5. mCherry+, DAB+, and 7 min irradiation; 6. mCherry+, PANI+, and 7 min irradiation; 7. miniSOG+, DAB−, and 7 min irradiation; 8. miniSOG+, DAB+, and 7 min irradiation; and 9. miniSOG+, PANI+, and 7 min irradiation. At DIV 14, neurons were infected with 1×10¹⁰ vg (viral genome) AAVdj-hSyn-miniSOG-T2A-mCherry or AAVdj-hSyn-mCherry (Except group 1). 3-5 days after infection when mCherry fluorescence was observed, DAB was added to a final concentration of 1 mM or aniline and PPD mixture was added to 0.5 mM in Tyrode's solution respectively with pH adjusted to 7.3-7.4 and osmolarity adjusted to 320-330 mOsm. Then the solution containing monomers was filtered with a 0.22 m filter unit. Solution containing monomers was added into one of the wells each time following the group settings and neurons were irradiated for 7 min with ˜5 mW/mm² epifluorescence blue light. The Tyrode's solution was replaced with the original Nb4 media of each well. After all the wells were irradiated and then incubated at 37° C. for 30 min for recovery, the cell viability test was then performed. For the cell viability test, the cells were washed 1-3 times in 1×PBS as needed. 1× Calcein AM/NucRed™ Dead 647 ReadyProbes™ Reagent solution was prepared by diluting the provided Calcein stocks 1:500 in Nb4 medium and adding two drops of NucRed® Dead 647 Reagent per milliliter of medium. The original medium was gently removed from the wells. Each well was washed three times with fresh Nb4 medium to remove loosely attached and dead cells. 100 μL of medium containing Calcein AM and NucRed™ Dead 647 ReadyProbes™ Reagent was added to each well. The cells were incubated at room temperature (22-26° C.) for 5-15 minutes, protected from light. The Calcein AM&NucRed™ solution was removed and the wells washed 2 times with fresh Nb4 medium. After the last wash, enough Tyrode's solution was added to cover the cells. The presence of the green Calcein (Ex: 485 nm and Em: 515 nm) or far-red NucRed™ Dead 647 ReadyProbes™ Reagent (Ex: 642 nm and Em: 661 nm) fluorescence in the illuminated area was assessed by fluorescence microscopy using the same imaging parameters for all wells.

### Characterization of Long-Term Modulation of Neuron Excitability and Viability

1×10⁵ primary cultures of cortical rat neurons were added to Nb4 medium and cultured on PDL-coated glass coverslips. The cultured neurons were infected with 1×10¹⁰ vg AAVdj-hSyn-miniSOG-T2A-mCherry or AAVdj-hSyn-mCherry at 14 days in vitro (DIV), and at 17 DIV the medium was switched to Tyrode's solution (150 mM NaCl, 4 mM KCl, 2 mM MgCl₂, 2 mM CaCl₂), 20 mM glucose, 10 mM HEPES; titrated to pH 7.35 with NaOH and adjusted osmolarity to 320-330) or Tyrode's solution containing 0.5 mM aniline and 0.5 mM PPD (PANI+) or 1.0 mM DAB (PDAB+). The original Nb4 medium from the cultures was collected and incubated in a humid culture incubator with 5% CO₂ at 37° C. for later use. The Tyrode's solutions were pre-filtered with 0.22 μm filter units. The cultured neurons were then exposed to 7 minutes of blue light irradiation (˜5 mW/mm²) to induce polymerization. Following the end of polymerization, cultured neurons were washed with pre-warmed Nb4 medium 3 times and then maintained in a humid culture incubator with 5% CO₂ at 37° C. in the original Nb4 medium until subsequent electrophysiological characterization. After additional incubation of 1 or 3 days, the cultured neurons were taken for whole-cell patch clamp characterization or cell viability assays according to the aforementioned methods.

### Synthesis of Biotin-DAB:

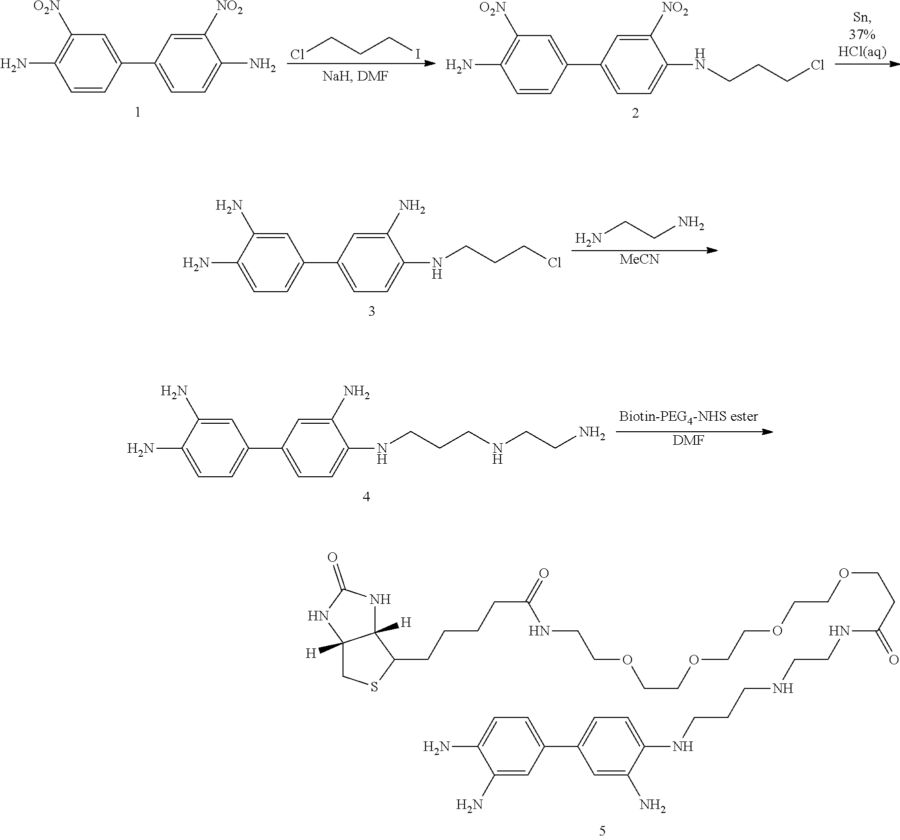

### General Synthesis Information:

All chemicals/reagents were commercially available and used without further purification. The NMR spectra for both ¹H and ¹³C were recorded from The Bruker Ascend 400 MHz NMR spectrometer at 298 K with the deuterated solvent (DMSO-d₆) as the lock and the residual solvents as the internal reference.

### Synthesis of Dinitro Benzidine-Cl (Compound 2)

To a solution of 3,3′-dinitrobenzidine (1 g, 3.646 mmol) in dry DMF, (20 ml) sodium hydride (131 mg, 5.47 mmol) was added in batches and the mixture was stirred at 0° C. for 30 mins. Then the mixture was added dropwise to a solution of 1-chloro-3-iodopropane (2.236 g, 10.939 mmol) in dry DMF (10 ml). The mixture was then stirred under ambient condition and room temperature overnight. After the reaction was complete, the solvent was removed with rotary evaporator and then purified by column chromatography on silica gel using DCM/Hexanes (10% to 50% of DCM) as the eluent to afford compound 2 as a red solid (450 mg, 35%). ¹H NMR (400 MHz, DMSO-d₆, 298 K) δ (ppm): 8.21 (d, J=34.7 Hz, 3H), 7.83 (dd, J=38.7, 8.8 Hz, 2H), 7.56 (s, 2H), 7.27-6.99 (m, 2H), 3.76 (s, 2H), 3.56 (s, 2H), 2.21-1.96 (m, 2H). ¹³C NMR (101 MHz, DMSO-d₆, 298 K) δ (ppm): 13C NMR (101 MHz, DMSO) δ 192.87, 151.34, 145.80, 144.46, 134.73, 134.09, 131.82, 130.84, 125.93, 122.81, 121.91, 120.60, 115.72, 43.50, 31.71.

### Synthesis of DAB-Cl (Compound 3)

Compound 2 (150 mg, 0.428 mmol) and Sn powder (300 mg, 2.53 mmol) were dissolved in 5 ml of 37% hydrochloric acid and stirred at room temperature for 30 mins. The mixture was then neutralized by saturated sodium bicarbonate solution, and then extracted with CHCl₃ for 3×20 ml each. The solvent was removed by rotary evaporator to afford compound 3 as light-yellow solid (100 mg, 81%). ¹H NMR (400 MHz, DMSO-d₆, 298 K) δ (ppm): 6.89-6.74 (m, 2H), 6.64 (q, J=14.2 Hz, 3H), 6.49 (dd, J=18.5, 8.2 Hz, 1H), 5.20 (s, 7H), 3.79 (t, J=6.5 Hz, 2H), 3.20-3.16 (m, 2H), 2.03 (h, J=7.0 Hz, 2H). ¹³C NMR (101 MHz, DMSO-d₆, 298 K) δ (ppm): 135.29, 134.96, 132.78, 131.69, 130.61, 116.95, 116.28, 115.97, 113.66, 112.67, 110.88, 55.41, 43.95, 41.07, 32.18.

### Synthesis of DAB-Amine (Compound 4)

Compound 3 (50 mg, 0.172 mmol) and ethylenediamine (1.0 g, 16.65 mmol) were dissolved in 2 ml of MeCN and the solution bubbled with nitrogen for 30 mins. The system was then heated to 60° C. and stirred under nitrogen atmosphere overnight. The solvent was then removed and the crude compounds purified by RP-HPLC (water/acetonitrile+0.10% formic acid) to afford compound 4 as an orange solid (40 mg, 74%). ¹H NMR (400 MHz, DMSO-d₆, 298 K, protonated) 8.39 (s, 3H), 6.73 (d, J=2.1 Hz, 1H), 6.69 (d, J=1.9 Hz, 1H), 6.64 (d, J=8.0 Hz, 1H), 6.55 (d, J=8.2 Hz, 1H), 6.49 (d, J=7.9 Hz, 1H), 6.42 (d, J=8.2 Hz, 1H), 6.36 (s, 8H), 3.09 (t, J=6.8 Hz, 2H), 2.98 (s, 2H), 2.91 (d, J=5.6 Hz, 2H), 2.81 (t, J=7.1 Hz, 2H), 1.83 (p, J=7.0 Hz, 2H). ¹³C NMR (101 MHz, DMSO-d₆, 298 K) δ (ppm): 166.58, 135.80, 135.53, 134.81, 133.82, 131.25, 130.86, 115.30, 112.61, 112.26, 110.61, 46.53, 46.39, 41.77, 38.19, 37.72, 27.75.

### Synthesis of Biotin-DAB (Compound 5)

A 100 mM stock solution of compounds 4 in anhydrous DMSO was mixed with a 250 mM stock solution of Biotin-PEG₄-NHS ester in a 3:1 ratio (v/v) and stirred overnight at room temperature; the resulting solution was used for in situ polymerization without further purification. (ESI-MS: [M+H]⁺: calculated m/z: 788.47, found m/z: 788.97).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for modulating neural activity using optogenetic polymerization, increasing current injection-evoked action potential firing in response to depolarizing stimuli and assembly of electroactive polymers on specified cellular membranes, or decreasing action potential firing in response to depolarizing stimuli, the method comprising
(a) expressing in a neuronal cell a vector comprising a mini Singlet Oxygen Generator (miniSOG) in the presence of monomers of 3,3′-diaminobenzidine (DAB) or aniline and N-phenyl-p-phenylenediamine; and
(b) irradiating at least a portion of the neuronal cell to induce polymerization of poly(3,3′-diaminobenzidine) or polyaniline, thereby modulating the neuronal activity.

2. The method of claim 1, wherein the method provides for
the spatiotemporal control of polymerization;
photopolymerization of DAB at nanometer-level spatial resolution; and/or
optical control of polymer assembly on or within the cell membrane.

3. The method of claim 1, wherein the miniSOG produces increased levels of singlet oxygen relative to other reactive oxygen species (ROS).

4. The method of claim 1, wherein the method does not reduce neuron viability.

5. The method of claim 1, wherein the method alters neuronal excitability.

6. The method of claim 1, wherein the miniSOG is expressed under the control of a CAG promoter.

7. The method of claim 1, wherein the vector is a viral vector.

8. The method of claim 7, wherein the viral vector is an adeno-associated viral expression vector (AAV) vector.

9. The method of claim 1, wherein the irradiation is at a wave length between about 425-500 nm.

10. The method of claim 1, wherein the irradiation is for about 5-8 or 9-15 minutes.

11. The method of claim 1, wherein the neuron is in vitro or in vivo.

12. An adeno-associated viral expression vector (AAV) comprising a CAG or human synapsin promoter driving expression of a miniSOG fused to a T2A ribosome skipping sequence.

13. A neuronal cell comprising the AAV of claim 12.

14. A method for modulating neural activity using optogenetic polymerization and assembly of electroactive polymers on specified cellular membranes, increasing current injection-evoked action potential firing in response to depolarizing stimuli, and/or decreasing action potential firing in response to depolarizing stimuli, the method comprising
(a) expressing in a neuronal cell an adeno-associated viral expression vector (AAV) comprising a CAG or human synapsin promoter driving expression of a miniSOG fused to a T2A ribosome skipping sequence in the presence of monomers of 3,3′-diaminobenzidine or aniline and N-phenyl-p-phenylenediamine; and
(b) irradiating the neuronal cell to induce polymerization of poly(3,3′-diaminobenzidine) or polyaniline, thereby modulating the neuronal activity.

15. The method of claim 1, wherein the method provides long term alterations in the electrophysiology of the neuron.

16. The method of claim 1, wherein the electrophysiological changes last for at least about 1 month, about 1-3 weeks, or about 1-3 days.

17. A method for modulating neural activity in a subject having a disorder associated with undesirable neural activity, the method comprising
(a) administering to a neuronal cell of the subject a vector comprising a mini Singlet Oxygen Generator (miniSOG) in the presence of monomers of 3,3′-diaminobenzidine or aniline and N-phenyl-p-phenylenediamine; and
(b) irradiating at least a portion of the neuronal cell to induce polymerization of poly(3,3′-diaminobenzidine) or polyaniline, thereby modulating the neuronal activity of the subject.

18. A method for modulating neural activity in a subject having a disorder associated with undesirable neural activity, the method comprising
(a) administering to a neuronal cell of the subject an adeno-associated viral expression vector (AAV) comprising a CAG or human synapsin promoter driving expression of a miniSOG fused to a T2A ribosome skipping sequence in the presence of monomers of 3,3′-diaminobenzidine or aniline and N-phenyl-p-phenylenediamine; and
(b) irradiating the neuronal cell to induce polymerization of poly(3,3′-diaminobenzidine) or polyaniline, thereby modulating the neuronal activity of the subject.

19. The method of claim 18, wherein the disorder is characterized by increased neuronal excitability, is a neurodegenerative disease, is chronic pain or epilepsy.

20. The method of claim 19, wherein the neuron is irradiated in the presence of aniline and N-phenyl-p-phenylenediamine.

21. A kit for use in any of the above methods, the kit comprising a vector comprising a mini Singlet Oxygen Generator (miniSOG) and monomers of 3,3′-diaminobenzidine and/or aniline and N-phenyl-p-phenylenediamine.
